SEQUENCE LISTING (7 sequences)

SEQ ID NO: 1 - DNA, 4711 bp, artificial sequence
  SEQ ID NO1 Expression Cassette
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 2 - DNA, 5984 bp, artificial sequence
  SEQ ID NO2 Expression Cassette
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctccccccctcccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcgggggggctgcgaggggaacaaaggctgcgtgcggggtgtgtgcgtggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcggggggacggggcagggcggggttcggcttctggcgtgtgaccggcggctctagaaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagatttttttatattttgttttgtgttattttttctttaacatccctaaaattttccttacatgttttactagccagatttttcctcctctcctgactactcccagtcatagctgtccctcttctcttatggagatc

SEQ ID NO: 3 - DNA, 5314 bp, artificial sequence
  SEQ ID NO3 Expression Cassette
ggtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcacaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatctagagtcggggcggccggccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccgagtgtcgataag

SEQ ID NO: 4 - DNA, 4678 bp, Artificial Sequence
  SEQ ID NO4 Expression Cassette
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatcgcggccgctcgagtctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 5 - DNA, 33765 bp, artificial sequence
  SEQ ID NO5, Maternal sequence of recombinant human adenovirus serotype 26 with E1 and E3 sites deleted and the site ORF6-Ad26 substituted for ORF6-Ad5
catcatcaataatataccccacaaagtaaacaaaagttaatatgcaaatgagcttttgaattttaacggttttggggcggagccaacgctgattggacgagaaacggtgatgcaaatgacgtcacgacgcacggctaacggtcgccgcggaggcgtggcctagcccggaagcaagtcgcggggctgatgacgtataaaaaagcggactttagacccggaaacggccgattttcccgcggccacgcccggatatgaggtaattctgggcggatgcaagtgaaattaggtcattttggcgcgaaaactgaatgaggaagtgaaaagcgaaaaataccggtccctcccagggcggaatatttaccgagggccgagagactttgaccgattacgtgggggtttcgattgcggtgtttttttcgcgaatttccgcgtccgtgtcaaagtccggtgtttatgtcacagatcagctggtttcctttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcggatatcttatgtgtaatgtaatttgactcctttgagcacgggctcagaatcgtcctcgtcgaacttgcagcagctgccacagctacaacagcccttcaggcagctacagcagctggtcatgcaacacagcatgattgtgaccatcacgatggcaatcagtccggcgataaagcccagccagatgtaccagggccacttgatgtactgctcgtacttccccagttcttgcaggtcgatcaggctctcgttcagattcttggccacctcgttcagccggtcgatctctttctggatgttcacgacgctggcattgattccgctgatgtcgcccaggtccacgtcggggcttgtgtggttcttaaagtacttatccagttcctctttgaagctgtccagctcgggctgcagagggtcgtacacggtattgttcacaatgccgatcacgacgtcgcagttgccagacacgaaggtgttgtcggtggtgatgatctggggctcgtagaagttccgctgggtcacgaaccaatgggtgccgttggacacgaacacgccttctctaggaaagtgggctttgccgtcgtggcagatggctggagcggtggtgaaattcttctcttgagcgggcacgtatgtcacgtgcagaaacaccacgccgtgaggggcagactgagggaagctcatcaggtggtagcccttgccgcaaaagtccactctcttgctctggcccagcacacactcagacatcttggtggcggccagattggcagaggctctaatctcggcggctctgatcagctgctgggtaacgtaggtctgcagggactgcagccttccggtgatcagtctgtcgatctgcacctcggcttccaccttgtccagtctgctcaggatgtcgttcagcacagagctgatggcgccgaagttggaggacagctgcttgaccagggtgttcagtgcctgggcattctggttgaccacgtcctgcagctttcccagggcgcttgctgtgctgctcaggctgtcctggatcttgccgatggcgctgttgaactggttggcgatcagcttctggttctcgtacagcacattctgggtcactccgatgccgttgaaccggtaggccatctgcatagcaaaggggatctgcagagcggcgccagctccaaatgtccagccgcttgtgattgtgccggccagcagggcagatgtgtactgggcgatcatctcatcggtcagcagtggtggcagcactgtcagtccgttaaacttctgggcgcaaatcagatccctggcggcaatgtcgcccagacaatcgccatactgcttgatgaagccggcgtcggccagtgtcactttgttgaacagcaggtcctcgatgaagctccgcttgctgggcttgctaggatcgggcagaatctggctgaaattgaagccgccgaagtccttgataggaggggtcttgtagatctgcttcacttgggcgaacacctcttgggtgttcttgtcctgttccacggcgatccctgtcagggctctattcagctgggtgcagaagctgccgtactgcagcagcaggttggagcactcggtggaatcgccgcagatgtacatggtgcagtccacgctggtcttggtcatggacacaggcaggatctctgtggtcacgctgatggtgaagttggtggggatagcgatagagttgttggagtaggccacgctgttctcggcgcccagagacattgtgtaggcaatgatgctctggctggccacagatctggcccgtctggggctgtttgtctgtgtctggtagctggcacagatgccagcgccgatggggatgtcgcactcgtagctattgttcacgtgctcggctccgatcagacagccggctctggtctgaaacacattgctgccggtggagtacacccgccatgtaggtgtcagctgatcggcgtgaatggccacgggcacttcggtacagttcacgtcctggtacagcactgccacctgattgctggtgttggtgccaggggtgatcacagacactccgccgaagctgcaaggggtgatgtccaggatttccagtgtctggggatctcgtacggcgtctgtggtatcggcaatatcccggccaaactgctggaatggcaggaacttcttgttgctctctgtcagcacgccggtgccggtcaggccgttgaagttgaagttcacgcatttgttcttcacgagattggtgcttttcttagggccgcacactgtggcaggggcatgcagcagttcgaagctcagcaccaccactctgtagggctgatagcccacgccatttgtgggctgaaagccgtaggactgcagtgggaagtagcagttgaagccttccacgccgttacaaggggtgctgccggcctgatagatctcggtggagatgtcccgctcgaagggcttcagattggacttccggaacagccggtacaggtaattgtagttgccgccgactttggagtccaggttgttgctgttccaggcaatcacacagccggtgaagtcgtcgggcagcttgtagttgtagtcggcgatcttgcctgtctgtccaggggcaatctgccgcacttcatctccccggatcacgaagctgtcggcgtacacgtttgtgaagcacaggtcgttcagcttggtaggggacacgccgtagcacttgaaggtgctgaagctggcggagttgtacagcacggagtagtcggccacgcaattgctgatccgcttccggttccaggcgtacacagaggcgaatctggtggcattgaacacctcgccgaaggggcacagattggtgatattggggaaccgcacgatggattcggtgggctgcacccggaagttgctggtctggtagatgcccttttccacggtgaaggacttcagggtgcactttgtctcgctcagaggatccagagcacaatccacggcgtcggtgatggtgccgttctcgttgtacttcagcaggaaggttctaggctgcaggtagcccacatagtaagcggcggcaccagctgtccatccgctgctgctatcgccaggtgtcaggtagcttctgtgcagggccagcagtgtctgaaaccgggtgatgttgatgccgatgggcagatccaccaggggttccagagcagagaagccctgaggcagatcccgcacgaggttgataggggtgtgcttgctgtagatcttgaagtagccgtcgatgttcttgaacacgaactcgcgcaggttcttgaagttgccctgcttgccttccaggtccatcaggaaaggctgggacacgtactcgaaggtgcagttgttggcgctgctgtacacccggaactcgctttccatccagctcttgttgttcttgtgatagtagacgcccaggaaggggtcgttgcagaactggaactcgcacactttgatgaccacgttggtggcgttgttcacgatcagcaggctctgggtcttgctgtccagtgtggtgccgaagatccagcctctgatgatgttggacttctcggtgctggcaaagtacaccccgtcgttgaagggcagcacggggttgtcgaatctcttggtgccattggtgccggacacgtggatggcgtggaaccaggtcacgttgctgaagaaaggcaggaacaggtcctgggtagagtgcagcacgctggatctgaacaccttgtcggggtagtacacgcctctggtaaagctgttggtgtaggctggaggcagctgggttcttgtggtcaggttcacacactgactagagactagtggcaataacacaaggaacacaaacatggtaccaagcttaggctcgagcggccgcgtcgacggtaccagatctctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaacagtgtttaaacgtatacctataaaggcgggtgtcttacgagggtctttttgcttttctgcagacatcatgaacgggactggcggggccttcgaaggggggctttttagcccttatttgacaacccgcctgccgggatgggccggagttcgtcagaatgtgatgggatcgacggtggatgggcgcccagtgcttccagcaaattcctcgaccatgacctacgcgaccgtggggaactcgtcgctcgacagcaccgccgcagccgcggcagccgcagccgccatgacagcgacgagactggcctcgagctacatgcccagcagcggtagtagcccctctgtgcccagttccatcatcgccgaggagaaactgctggccctgctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgtgggtggaggtagcaccactgcatggcctcgtgctctggggtcgtgttgtagatgatccagtcatagcaggggcgctgggcgtggtgctggatgatgtccttgaggaggagactgatggccacggggagccccttggtgtaggtgttggcaaaacggttgagctgggagggatgcatgcggggggagatgatgtgcagtttggcctggatcttgaggttggcgatgttgccacccagatcccgccgggggttcatgttgtgcaggaccaccagaacggtgtagcccgtgcacttggggaacttgtcatgcaacttggaagggaatgcgtggaagaatttggagacgcccttgtgcccgcccaggttttccatgcactcatccatgatgatggcaatgggcccgtgggctgcggctttggcaaagacgtttctggggtcagagacatcgtaattatgctcctgggtgagatcatcataagacattttaatgaatttggggcggagggtgccagattgggggacgatggttccctcgggccccggggcgaagttcccctcgcagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcttgtctctgacttggaggttttcccggacgagctcgccgaggaggcggtccccgcccagcgagagaagctcttgcagggaagcaaagtttttcaggggcttgagcccgtcggccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagggtggtctccgtcacggtgaaggggtgggccgcgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaggcgtccgctccgcagtgggcgcagacggtctcgcactcgactagccaggtgagctcgggctgctcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcagaccactctgagacgaaggcgcgcgtccacgccaagacaaaggaggccacgtgcgaggggtagcggtcgttgtccaccagggggtccaccttttccacggtatgcaggcacatgtccccctcctccgcatccaagaaggtgattggcttgtaggtgtaggccacgtgacctggggttcccgacgggggggtataaaagggggcgggtctgtgctcgtcctcactctcttccgcgtcgctgtccacgagcgccagctgttggggtaggtattccctctcaagagcgggcatgacctcggcactcaggttgtcagtttctagaaacgaggaggatttgatgtgggcctgccctgccgcgatgctttttaggagactttcatccatctggtcagaaaagactatttttttattgtcaagcttggtggcgaaggagccatagagggcgtttgagagaagcttggcgatggatctcatggtctgatttttgtcacggtcggcgcgctccttggccgcgatgttgagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtctgccgcccttgcgcgagcagaacgggggcagcacatcaagcagatgctcgtcaggggggtccgcatcgatggtgaagatgcccggacagagttccttgtcaaaataatcgatttttgaggatgcatcgtccaaggccatctgccactcgcgggcggccagcgctcgctcgtaggggttgaggggcggaccccaaggcatgggatgcgtgagggcggaggcgtacatgccgcagatgtcatagacatagatgggctccgagaggatgccgatgtaggtgggatagcagcgccccccgcggatgcttgcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtgaggcaggcggaccgagtcgcggatgaagtgcgcgtaggagtcttgcagcttggcgacgagctcggcggtgacgaggacgtccatggcgcagtagtccagcgtttcgcggatgatgtcataactcgcctctcctttcttctcccacagctcgcggttgagggcgtattcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcaaaggtgtccctgaccatgactttcaagaactggtacttgaagtccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcaaagccgttgatgttgtgcccgacgatgtagagttccatgaatcgcgggcggcctttgatgtgcggcagctttttgagctcctcgtaggtgaggtcctcggggcattgcaggccgtgctgctcgagcgcccactcctggagatgtgggttggcttgcatgaaggaagcccagagctcgcgggccatgagggtctggagctcgtcgcgaaagaggcggaactgctggcccacggccatcttttctggggtgacgcagtagaaggtgagggggtcccgctcccagcgatcccagcgtaaacgcacggcgagatcgcgagcgagggcgaccagctctgggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggtcgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctctgcgtcattgacggcggcctgacgcaggatctcttgcacgtcgcccgagttgtcctggtaggcgatctcggacatgaactgttcgatctcctcctcctggagatcgccgcggcccgcgcgctccacggtggcggcgaggtcattggagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcaaagacggcgtagttgcgcaggcgctggaagaggtagttgagggtggtggcgatgtgctcggtgacgaagaagtacatgatccagcggcgcaggggcatctcgctgatgtcgccgatggcttccagcctttccatggcctcgtagaagtccacggcgaagttgaaaaactgggcgttgcgggccgagaccgtgagctcgtcttccaggagccggatgagttcggcgatggtggcgcgcacctcgcgctcgaaatccccgggggcctcctcctcttcctcttcttccatgacgacctcttcttctatttcttcctctgggggcggtggtggtggcgggggccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccattgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgcggacgctgttagaattgcggttgctgatgatgtaattgaagtaggcgtttttgaggcggcggatggtggcgaggaggaccaggtccttgggtccagcttgctggatgcggagccgctcggccatgccccaggcctggccctgacaccggctcaggttcttgtagtagtcatgcatgagcctctcaatgtcatcactggctgaggcggagtcttccatgcgggtgaccccgacgcccctgagcggctgcacgagcgccaggtcggcgacgacgcgctcggcgaggatggcctgttgcacgcgggtgagggtgtcctggaagtcgtccatgtcgacgaagcggtgataggccccggtgttgatggtgtaggtgcagttggccatgagcgaccagttgacggtctgcaggcctggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgctggccgaggggggtcgctagacttgaaagcgaccgaaaaccctgccgggtagtggctcgcgcccgtagtctggagaagcatcgccagggttgagtcgcggcagaacccggttcgaggacggccgcggcgagcgggacttggtcaccccgccgatataaagacccacagccagccgacttctccagttacgggagcgagcccccttttttctttttgccagatgcatcccgtcctgcgccaaatgcgtcccacccccccggcgaccaccgcgaccgcggccgtagcaggcgccggcgctagccagccaccacagacagagatggacttggaagagggcgaagggctggcaagactgggggcgccgtccccggagcgacatccccgcgtgcagctgcagaaggacgtgcgcccggcgtacgtgcctacgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcacgcgcgcacgtggcggcagccaacctggtgacggcctacgagcagacggtgaagcaggagcgcaacttccaaaagagtttcaacaaccacgtgcgcaccctgatcgcgcgcgaggaggtggccctgggcctgatgcacctgtgggacctggcggaggccatcgtgcagaacccggacagcaagcctctgacggcgcagctgttcctggtggtgcagcacagcagggacaacgaggcgttcagggaggcgctgctgaacatcgccgagcccgagggtcgctggctgctggagctgattaacatcttgcagagcatcgtagtgcaggagcgcagcctgagcctggccgagaaggtggcggcgatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtaccgcaacgaccgcatccacaaggccgtgagcacgagccggcggcgcgagctaagcgaccgcgagctgatgctgagtctgcgccgggcgctggtagggggcgccgccggcggcgaggagtcctacttcgacatgggtgcggacctgcattggcagccgagccggcgcgccttggaggccgcctacggttcagaggacttggatgaggaagaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggatcggctggtgacggacgtgcgcgaggccgtggcgcagcgcgagcggttcaagaacgagggcctgggctcgctggtggcgctgaacgccttcctggcaacgcagccggcgaacgtgccgcgcgggcaggacgattacaccaactttatcagcgcgctgcggctgatggtgaccgaggtgccccagagcgaggtgtaccagtctggcccggactactttttccagacgagccggcagggcttgcagacggtgaacctgagccaggctttcaagaatctgcgcgggctgtggggcgtgcaggcgcccgtgggcgaccggtcaacggtgagcagcttgctgacgcccaactcgcggctgctgctgctgctgatcgcgcccttcaccgacagcggcagcgtgaaccgcaactcgtacctgggccatctgctgacgctgtaccgcgaggccataggccaggcgcaggtggacgagcagaccttccaggagatcactagcgtgagccgcgcgctggggcagaacgacaccgacagtctgagggccaccctgaactttttgctgaccaatagacagcagaagatcccggcgcagtacgcactgtcggccgaggaggaaaggattctgagatatgtgcagcagagcgtagggctgttcctgatgcaggagggtgccacccccagcgccgcgctggacatgaccgcgcgcaacatggaacctagcatgtacgccgccaaccggccgttcatcaataagctgatggactacttgcaccgcgcggcggccatgaacacggactactttaccaacgccatcctgaacccgcactggctcccgccgccggggttctacacgggcgagtacgacatgcccgaccccaacgacgggttcctgtgggacgacgtggacagcgcggtgttctcgccgacctttcaaaagcgccaggaggcgccgccgagcgagggcgcggtggggaggagcccctttcctagcttagggagtttgcatagcttgccgggctcggtgaacagcggcagggtgagccggccgcgcttgctgggcgaggacgagtacctgaacgactcgctgctgcagccgccgcgggccaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggtcaacccgttcgcgcatctgcagcccaaactggggcgacggatgttttgaatgaaataaaactcaccaaggccatagcgtgcgttctcttccttgttagagatgaggcgcgcggtggtgtcttcctctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacactaacatgcccaatgtgaacgagtacatgttcaccagcaagtttaaggcgcgggtgatggtgtctaggaagcatccagagggggtagttgaaacagatttgagtcaggataagcttgaatatgagtggtttgagtttaccctgcccgagggaaacttttccgagaccatgaccatagacctgatgaacaacgccatcttggaaaactacttgcaagtggggcggcagaatggcgtgctggagagcgatatcggagtcaagtttgacagcagaaatttcaagctgggctgggacccggtgaccaagctggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaggatctagaaggtggcaacatccccgccctccttgatgtgcccaagtacttggaaagcaagaagaaagttgaagacgaaactaaaaatgcagctgcggccacagccgatacaaccactaggggtgatacatttgcaactccagcgcaagagacagcagctgataagaaggtagaagtcttgcccattgaaaaggatgagagtggtagaagttacaacctgatccaggggacccacgacacgctgtaccgcagttggtacctgtcctatacctacggggaccccgagaagggggtgcagtcgtggacgctgctcaccaccccggacgttacctgcggcgcggagcaagtctactggtcactgccggacctcatgcaagaccccgtcaccttccgctccacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaaatgtctattctcatctcgcccagcaataacaccggctggggtcttactagacccagcaccatgtacggaggagccaagaagcgctcccagcagcaccccgtccgcgtccgcggccacttccgcgctccctggggcgcttacaagcgcgggcggacttccaccgccgtgcgcaccaccgtcgacgacgtcatcgactcggtggtcgccgacgcgcgcaactacactcccgccccctccaccgtggacgcggtcatcgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgctgccactgcacccacccccgcaggcaggactcgcagacgagcggccgccgccgccgctgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaccccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcaaaccgtggacattcagatccccaccgacatggatgtcgacaaaaaaccctcgaccagcatcgaggtgcaaaccgacccctggctcccagcctccaccgctaccgtctccacttctaccgccgccacggctaccgagcctcccaggaggcgaagatggggcgccgccagccggctgatgcccaactacgtgttgcatccttccatcatcccgacgccgggctaccgcggcacccggtactacgccagccgccggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaattcgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcggctttctgcccgcgctcatccccataatcgccgcggccattggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccaaaacttccagcagaaggtggtggacgggctggcctcgggcattaacggggtggtggacatcgcgaaccaggccgtgcagcgcgagataaacagccgcctggacccgcggccgcccacggtggtggagatggaagatgcaactcttccgccgcccaaaggcgaaaagcggccgcggcccgacgcggaggagacgatcctgcaggtggacgagccgccctcgtacgaggaggccgtcaaggccggcatgcccaccacgcgcatcatcgcgccgctggccacgggtgtaatgaaacccgccacccttgacctgcctccaccacccgcgcccgctccaccgaaggcaactccggttgtgcaggcccccccggtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagcctggctcccaagggtgcccccaatcccagtcagtgggaaacaaaagaaaagcaaggaactactggaggagtgcagcaagaaaaagatgtcacaaaaacatttggtgtggctgccaccggcggaattaatataacaaaccagggtctgttactaggaactgacgaaaccgctgagaatggcaaaaaagacatttatgcagacaagactttccagccagaacctcaagttggagaagaaaactggcaggaaaatgaagccttctatggaggaagggctcttaaaaaggacactaaaatgaaaccatgctatggatcttttgctagacctactaatgagaaaggaggtcaggcaaagttcaaaccagttaatgaaggagaacaacctaaagatctggatatagattttgcttactttgacgtccctggcggaagtcctccagcaggtggtagtggggaagaatacaaagcagatataattttgtacactgaaaatgttaatcttgaaacaccagacactcatgtggtttacaagccaggaacttcagataacagttcagaaatcaatctggttcagcagtccatgccaaacagacccaactacattggctttagggacaactttgtaggtctcatgtattacaacagcaccggaaatatgggtgtgctggctggtcaggcttctcagttgaacgctgtggtcgacttgcaagacagaaacaccgagttatcttaccagctattgctagattctctgggtgacagaaccagatactttagcatgtggaactctgcggtggacagttacgatccagatgtcaggatcattgaaaatcacggtgtggaagatgaacttccaaactattgcttcccattgaatggcactggaaccaattccacttatcaaggtgtaaagattacaaatggtaatgatggtgctgaagaaagtgagtgggagaaagacgatgcaatttctagacaaaaccaaatctgcaagggcaatgtctacgccatggagatcaacctgcaggccaacctgtggaagagttttctgtactcgaacgtggccctgtacctgcccgactcctacaagtacacgccggccaacgtcaagctgcccgccaacaccaacacctacgagtacatgaacggccgcgtggtagccccatccctggtggacgcctacatcaacatcggcgcccgctggtcgttggaccccatggacaacgtcaaccccttcaaccaccaccgcaatgcgggcctgcgctaccgctccatgctgctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctatacgccactttcttccccatggcgcacaacaccgcttcaaccttggaagccatgctgcgcaacgacaccaacgaccagtccttcaacgactacctctcggccgccaacatgctctaccccatcccggccaaggccaccaacgtgcccatctccatcccatcgcgcaactgggccgccttccgcggctggagtttcacccggctcaagaccaaggaaactccttccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaataactcgggcttcaccggctacctcgcacccaccatgcgccaggggcagccctaccccgccaacttcccctacccgctcatcggtcagacagccgtgccctccgtcacccagaaaaagttcctctgcgacagggtcatgtggcgcatcccattctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtacaccagccgcaccgcggcgtcatcgaggccgtctacctgcgcacgcccttctccgccggcaacgccaccacctaagcatgagcggctccagcgaacgagagctcgcggccatcgtgcgcgacctgggctgcgggccctactttttgggcacccacgacaagcgcttcccgggctttctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgcagcgccctggcctcctcgcccgaccgctgtctcagcctcgagcagtccactcagaccgtgcaggggcccgactccgccgcctgcggactcttctgttgcatgttcttgcatgccttcgtgcactggcccgaccgacccatggacggaaaccccaccatgaacttgctgacgggggtgcccaacggcatgctacaatcgccacaggtgctgcccaccctcaggcgcaaccaggaggaactctaccgcttcctcgcgcgccactccccttactttcgctcccaccgcgccgccatcgaacacgccaccgcttttgacaaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggctgccacttgaactcggggatcaccagtttgggcactggggtctcggggaaggtctcgctccacatgcgccggctcatctgcagggcgcccagcatgtccggggcggagatcttgaaatcgcagttggggccggtgctctgcgcgcgcgagttgcggtacacggggttgcagcactggaacaccatcagactggggtacttcacactagccagcacgctcttgtcgctgatctgatccttgtccagatcctcggcgttgctcaggccgaacggggtcatcttgcacagctggcgtcccaggaagggcacgctctgaggcttgtggttacactcgcagtgcacgggcatcagcatcatccccgcgccgcgctgcatattcgggtagagggccttgacaaaggccgcgatctgcttgaaagcttgctgggccttggccccctcgctgaaaaacaggccgcagctcttcccgctgaactggttattcccacacccggcatcctgcacgcagcagcgcgcgtcatggctggtcagttgcaccacgctccgtccccagcggttctgggtcaccttagccttgctgggctgctccttcaacgcgcgctgcccgttctcgctggtcacatccatctccaccacgtggtccttgtggatcatcatcgtcccgtgcagacacttgagctggccttccacctcggtgcagccgtgatcccacagggcgcaaccggtgcactcccagttcttgtgcgcaatcccgctgtggctgaagatgtaaccttgcaacatgcggcccatgatggtgctaaatgctttctgggtggtgaaggtcagttgcatcccgcgggcctcctcgttcatccaggtctggcacatcttctggaagatctcggtctgctcgggcatgagcttgtaagcatcgcgcaggccgctgtcgacgcggtagcgttccatcagcacgttcatggtatccatgcccttctcccaggacgagaccagaggcagactcagagggttgcgtacgttcaggacaccgggggtcgcgggctcgacgatgcgttttccgtccttgccttccttcaatagaaccggcggctggctgaatcccactcccacgatcacggcatcttcctggggcatctcttcgtcggggtctaccttggtcacatgcttggtctttctggcttgcttcttttttggagggctgtccacggggagcacgtcctcctcggaagacccggagcccacccgctgatactttcggcgcttggtgggcagaggaggtggcggcgaggggctcctctcctgctccggcggatagcgcgccgacccgtggccccggggcggagtggcctctcggcccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacctgcagcgccagtccctcatcctccgggacgccctggccgaccggagcgaaacccccctcagcgtcgaggagctgtgtcgggcctacgagctcaacctcttctcgccgcgcgtaccccccaaacgccagcccaacggcacctgcgagcccaacccgcgtctcaacttctatcccgtctttgcggtccccgaagccctcgccacctatcacatctttttcaagaaccaaaagatccccgtctcctgccgcgccaaccgcaccagcgccgacgcgctcctcgctctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagctcatcatgccccacatcgaggccctcgatgaaagtcaggagcagcgccccgaggacgcccggcccgtggtcagcgacgagatgctcgcgcgctggctcgggacccacgacccccaggctttggaacagcggcgcaagctcatgctggccgtggtcctggttaccctcgagctggaatgcatgcgccgcttcttcagcgaccccgagaccctgcgcaaggtcgaggagaccctgcactacactttcagacacggtttcgtcaggcaggcctgcaagatctccaacgtggagctgaccaacctggtctcctgcctggggatcctgcacgagaaccgcctggggcagaccgtgctccactctaccctgaagggcgaggcgcggcgggactatgtccgcgactgcgtctttctatttctttgccacacatggcaagcagccatgggcgtgtggcaacagtgtctcgaggacgataacctgaaggagctggacaagcttcttgctagaaatcttaaaaagctgtggacgggcttcgacgagcgcaccgtcgcctcggacctggccgagatcgtgttccccgagcgcctgaggcagacgctgaaaggcgggctgcccgacttcatgagccagagcatgttgcaaaactaccgcactttcattctcgagcgatctgggatgctgcccgccacctgcaacgctttcccctccgactttgtcccgctgagctaccgcgagtgtcccccgccgctgtggagccactgctacctcttgcagctggccaactacatcgcctaccactcggacgtgatcgaggacgtgagcggcgaggggctgctcgagtgccactgccgctgcaacctgtgctccccgcaccgctccctggtctgcaacccccagctactaagcgagacccaggtcatcggtacctttgagctgcaaggtccgcaggagtccaccgctccgctgaaactcacgccggggttgtggacttccgcgtacctgcgcaaatttgtacccgaggactaccacgcccacgagataaagttcttcgaggaccaatcgcgtccgcagcacgcggatctcacggcctgcgtcatcacccagggcgcaatcctcgcccaattgcacgccatccaaaaatcccgccaagagtttcttctgaaaaagggtagaggggtctacctggacccccagacgggcgaagtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagccccgccggtcacggatacaacctccgcagctccggccaagcctcctcgtagatgggatcgagtgaagggtgacggtaagcacgagcggcagggctaccgatcatggagggcccacaaagccgcgatcatcgcctgcttgcaagactgcggggggaacatcgctttcgcccgccgctacctgctcttccaccgcggggtaaacatcccccgcaacgtgttgcattactaccgtcaccttcacagctaagaaaaagcaagtaaaaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccaaatgggcctggccgcgggcgcctcccaggactactccacccgcatgaactggctcagtgccggcccctcgatgatctcacgggtcaacggggtccgcagtcatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcgatcggtctgcgaccggacggagtgttccaactagccggagccgggagatcctccttcactcccaaccaggcctacctgaccttgcagagcagctcttcggagcctcgctccggaggcatcggaaccctccagtttgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtccaaccccgaagttgcatgagaatttttgttgtactctttgtggtgagtttaataaaagctaaactcttgcaatactctggaccttgtcgtcgtcaactcaacgagaccgtctacctcaccaaccagactgaggtaaaactcacctgcagaccacacaagacctatatcatctggttcttcgagaacacctcatttgcagtctccaacactcactgcaacgacggtgttgaacttcccaacaacctttccagtggactgagttacgatacacatagagctaagctcgtcctctacaatccttttgtagagggaacctaccagtgccagagcggaccttgtactcacaccttccatttggtgaacgtcaccagcagcagcaacagctcagaaactaaccttccttctgatactaacaaacctcgtttcggaggtgagctaaggcttcccccttctgaggagggggttagcccatacgaagtggtcgggtatttgattttaggggtggtcctgggtgggtgcatagcggtgctagtcgagatggacggccaggcctccgagcagcgcatcctgcaactgcgcgtccgtcagcagcaggagcgtgccgccaaggagctcctcgatgccatcaacatccaccagtgcaagaagggcatcttctgcctggtcaaacaggcaaagatcacctacgagctcgtgtccggcggcaagcagcatcgcctcgcctatgagctgccccagcagaagcagaagttcacctgcatggtgggcgtcaaccccatagtcatcacccagcagtcgggcgagaccagcggctgcatccactgctcctgcgaaagccccgagtgcatctactccctgctcaagaccctttgcggactccgcgacctcctccccatgaactgatgttgattaaaagcccaaaaaccaatcagccccttccccctaatcattcaataaagatcacttacttgaaatctgaaagtatgtctctggtgtagttgttcagcagcacctcggtaccctcctcccagctctggtactccagtccccggcgggcggcgaacttcctccacaccttgaaagggatgtcaaattcctggtccacaattttcattgtcttccctctcagatggcaaagaggctccgggtggaagatgacttcaaccccgtctacccctatggctacgcgcggaatcagaatatccccttcctcactcccccctttgtctcctccgatggattcaaaaacttcccccctggggtcctgtcacttaaactggctgatccaatcaccatcaacaatggggatgtctcacttaaggtgggagggggacttgctgtagagcaacagactggtaacctaagcgtaaaccctgatgcacccttgcaagttgcaagtgataagctacagcttgctctggctcctccattcgaggtcagagatggaaagcttgctttaaaggcaggtaatggattaaaagtactagataattccattactggattgactggattattgaatacacttgtggtattaactggaaggggaataggaacggaggaattaaaaaatgacgatggtgtaacaaacaaaggagtcggcttgcgtgtaagacttggagatgacggcgggctgacatttgataaaaagggtgatttagtagcctggaataaaaaagatgacaggcgcaccctgtggacaacccctgacacatctccaaattgcaaaatgagtacagaaaaggattctaaacttacgttgacacttacaaagtgtggaagtcaggttctgggaaatgtatctttacttgcagttacaggtgaatatcatcaaatgactgctactacaaagaaggatgtaaaaatatctttactatttgatgagaatggaattctattaccatcttcgtcccttagcaaagattattggaattacagaagtgatgattctattgtatctcaaaaatataataatgcagttccattcatgccaaacctgacagcttatccaaaaccaagcgctcaaaatgcaaaaaactattcaagaactaaaatcataagtaatgtctacttaggtgctcttacctaccaacctgtaattatcactattgcatttaatcaggaaactgaaaatggatgtgcttattctataacatttaccttcacttggcaaaaagactattctgcccaacagtttgatgttacatcttttaccttctcatatcttacccaagagaacaaagacaaagactaataaaatgttttgaactgaatttatgaatctttatttatttttacaccagcacgggtagtcagtttcccaccaccagcccatttcacagtgtaaacaattctctcagcacgggtggccttaaacaggtcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaggtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaaggagctcgactgttcctcggtggacattgaaatggattctcttgcgtaccttgtcgtacttctgccagcagaaagtggctcgggaacagcagatacctttcctcctgctgtccttccgctgctgacgctcagtcatccaactgaagtacagccattcccgcaggttctccagcagctcctgtgcatctgatgaaacaaaagtcccgtcgatgcggattccccttaaaacatcagccaggacattgtaggccatcccaatccagttaatgcatcctgatctatcatgaagaggaggtgggggaagaactggaagaaccatttttattccaagcggtctcgaaggacgataaagtgcaagtcacgcaggtgacagcgttccccgccgctgtgctggtggaaacagacagccaggtcaaaacccactctattttcaaggtgctcgactgtggcttcgagcagtggctctacgcgtacatccagcataagaatcacattaaaggctggacctccatcgatttcatcaatcatcaggttacactcattcaccatccccaggtaattctcatttttccagccttggattatttctacaaattgttggtgtaagtccactccgcacatgtggaaaagttcccacagcgccccctccactttcataatcaggcagaccttcatattagaaacagatcctgctgctccaccacctgcagcgtgttcaaaacaacaagattcaatgaggttctgccctctgccctcagctcacgtctcagcgtcagctgcaaaaagtcactcaagtcctcagccactacagctgacaattcagagccagggctaagcgtgggactggcaagcgtgagtgagtaccacccaaaaactgcatgctggaataagctctctttgtgtcaccggtgatgccttccaataggtgagtgataaagcgaggtagtttttctttaatcatttgagtaatagaaaagtcctctaaataagtcactaggaccccaggaaccacaatgtggtagctgacagcgtgtcgctcaagcatggttagtagagatgagagtctgaaaaacagaaagcatgcactaaaccagagttgccagtctcactgaaggaaaaatcactctctccagcagcaaagtgcccactgggtggccctctcggacatacaaaaatcgatccgtgtggttaaagagcagcacagttagctcctgtcttctcccagcaaagatcacatcggactgggttagtatgcccctggaatggtagtcattcaaggccataaatctgccttggtagccattaggaatcagcacgctcactctcaagtgaaccaaaaccaccccatgcggaggaatgtggaaagattctgggcaaaaaaaggtatatctattgctagtcccttcctggacgggagcaatccctccagggctatctatgaaagcatacagagattcagccatagctcagcccgcttaccagtagacagagagcacagcagtacaagcgccaacagcagcgactgactacccactgacccagctccctatttaaaggcaccttacactgacgtaatgaccaaaggtctaaaaaccccgccaaaaaaacacacacgccctgggtgtttttcgcgaaaacacttccgcgttctcacttcctcgtatcgatttcgtgactcaacttccgggttcccacgttacgtcacttctgcccttacatgtaactcagccgtagggcgccatcttgcccacgtccaaaatggcttccatgtccggccacgcctccgcggcgaccgttagccgtgcgtcgtgacgtcatttgcatcaccgtttctcgtccaatcagcgttggctccgccccaaaaccgttaaaattcaaaagctcatttgcatattaacttttgtttactttgtggggtatattattgatgatg

SEQ ID NO: 6 - DNA, 34808 bp, Artificial Sequence
  SEQ ID NO6, Maternal sequence of recombinant simian adenovirus serotype 25with E1 and E3 sites deleted
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatacatggcccgaaaggagcgatgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 7 - DNA, 34742 bp, artificial sequence
  SEQ ID NO7, Maternal sequence of recombinant human adenovirus serotype 5 with E1 and E3 sites deleted
ccatcttcaataatatacctcaaacttttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttgcgatgcatgcgcgttgtcaaatatgagctcacaatgcttccatcaaacgagttggtgctcatggcggcggcggctgctgcaaaacagatacaaaactacataagacccccaccttatatattctttcccacccttaaccacgcccagatccgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagactcgagcggccgcgatatcttatgtgtaatgtaatttgactcctttgagcacgggctcagaatcgtcctcgtcgaacttgcagcagctgccacagctacaacagcccttcaggcagctacagcagctggtcatgcaacacagcatgattgtgaccatcacgatggcaatcagtccggcgataaagcccagccagatgtaccagggccacttgatgtactgctcgtacttccccagttcttgcaggtcgatcaggctctcgttcagattcttggccacctcgttcagccggtcgatctctttctggatgttcacgacgctggcattgattccgctgatgtcgcccaggtccacgtcggggcttgtgtggttcttaaagtacttatccagttcctctttgaagctgtccagctcgggctgcagagggtcgtacacggtattgttcacaatgccgatcacgacgtcgcagttgccagacacgaaggtgttgtcggtggtgatgatctggggctcgtagaagttccgctgggtcacgaaccaatgggtgccgttggacacgaacacgccttctctaggaaagtgggctttgccgtcgtggcagatggctggagcggtggtgaaattcttctcttgagcgggcacgtatgtcacgtgcagaaacaccacgccgtgaggggcagactgagggaagctcatcaggtggtagcccttgccgcaaaagtccactctcttgctctggcccagcacacactcagacatcttggtggcggccagattggcagaggctctaatctcggcggctctgatcagctgctgggtaacgtaggtctgcagggactgcagccttccggtgatcagtctgtcgatctgcacctcggcttccaccttgtccagtctgctcaggatgtcgttcagcacagagctgatggcgccgaagttggaggacagctgcttgaccagggtgttcagtgcctgggcattctggttgaccacgtcctgcagctttcccagggcgcttgctgtgctgctcaggctgtcctggatcttgccgatggcgctgttgaactggttggcgatcagcttctggttctcgtacagcacattctgggtcactccgatgccgttgaaccggtaggccatctgcatagcaaaggggatctgcagagcggcgccagctccaaatgtccagccgcttgtgattgtgccggccagcagggcagatgtgtactgggcgatcatctcatcggtcagcagtggtggcagcactgtcagtccgttaaacttctgggcgcaaatcagatccctggcggcaatgtcgcccagacaatcgccatactgcttgatgaagccggcgtcggccagtgtcactttgttgaacagcaggtcctcgatgaagctccgcttgctgggcttgctaggatcgggcagaatctggctgaaattgaagccgccgaagtccttgataggaggggtcttgtagatctgcttcacttgggcgaacacctcttgggtgttcttgtcctgttccacggcgatccctgtcagggctctattcagctgggtgcagaagctgccgtactgcagcagcaggttggagcactcggtggaatcgccgcagatgtacatggtgcagtccacgctggtcttggtcatggacacaggcaggatctctgtggtcacgctgatggtgaagttggtggggatagcgatagagttgttggagtaggccacgctgttctcggcgcccagagacattgtgtaggcaatgatgctctggctggccacagatctggcccgtctggggctgtttgtctgtgtctggtagctggcacagatgccagcgccgatggggatgtcgcactcgtagctattgttcacgtgctcggctccgatcagacagccggctctggtctgaaacacattgctgccggtggagtacacccgccatgtaggtgtcagctgatcggcgtgaatggccacgggcacttcggtacagttcacgtcctggtacagcactgccacctgattgctggtgttggtgccaggggtgatcacagacactccgccgaagctgcaaggggtgatgtccaggatttccagtgtctggggatctcgtacggcgtctgtggtatcggcaatatcccggccaaactgctggaatggcaggaacttcttgttgctctctgtcagcacgccggtgccggtcaggccgttgaagttgaagttcacgcatttgttcttcacgagattggtgcttttcttagggccgcacactgtggcaggggcatgcagcagttcgaagctcagcaccaccactctgtagggctgatagcccacgccatttgtgggctgaaagccgtaggactgcagtgggaagtagcagttgaagccttccacgccgttacaaggggtgctgccggcctgatagatctcggtggagatgtcccgctcgaagggcttcagattggacttccggaacagccggtacaggtaattgtagttgccgccgactttggagtccaggttgttgctgttccaggcaatcacacagccggtgaagtcgtcgggcagcttgtagttgtagtcggcgatcttgcctgtctgtccaggggcaatctgccgcacttcatctccccggatcacgaagctgtcggcgtacacgtttgtgaagcacaggtcgttcagcttggtaggggacacgccgtagcacttgaaggtgctgaagctggcggagttgtacagcacggagtagtcggccacgcaattgctgatccgcttccggttccaggcgtacacagaggcgaatctggtggcattgaacacctcgccgaaggggcacagattggtgatattggggaaccgcacgatggattcggtgggctgcacccggaagttgctggtctggtagatgcccttttccacggtgaaggacttcagggtgcactttgtctcgctcagaggatccagagcacaatccacggcgtcggtgatggtgccgttctcgttgtacttcagcaggaaggttctaggctgcaggtagcccacatagtaagcggcggcaccagctgtccatccgctgctgctatcgccaggtgtcaggtagcttctgtgcagggccagcagtgtctgaaaccgggtgatgttgatgccgatgggcagatccaccaggggttccagagcagagaagccctgaggcagatcccgcacgaggttgataggggtgtgcttgctgtagatcttgaagtagccgtcgatgttcttgaacacgaactcgcgcaggttcttgaagttgccctgcttgccttccaggtccatcaggaaaggctgggacacgtactcgaaggtgcagttgttggcgctgctgtacacccggaactcgctttccatccagctcttgttgttcttgtgatagtagacgcccaggaaggggtcgttgcagaactggaactcgcacactttgatgaccacgttggtggcgttgttcacgatcagcaggctctgggtcttgctgtccagtgtggtgccgaagatccagcctctgatgatgttggacttctcggtgctggcaaagtacaccccgtcgttgaagggcagcacggggttgtcgaatctcttggtgccattggtgccggacacgtggatggcgtggaaccaggtcacgttgctgaagaaaggcaggaacaggtcctgggtagagtgcagcacgctggatctgaacaccttgtcggggtagtacacgcctctggtaaagctgttggtgtaggctggaggcagctgggttcttgtggtcaggttcacacactgactagagactagtggcaataacacaaggaacacaaacatggtaccagatctctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattacagtattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcgctgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgccggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggcggacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgccagctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaacccatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatgtcgacccccggtcccccacccagtcccccgaggaggtccgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccactacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggatgcggtggccatccaccagtgcaagagaggcatcttctgcctggtgaaacaggccaagatctcctacgaggtcactccaaacgaccatcgcctctcctacgagctcctgcagcagcgccagaagttcacctgcctggtcggagtcaaccccatcgtcatcacccagcagtctggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcgtgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtaaataaatctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

(19) United States
(12) Patent Application Publication
(10) Pub. No.: US 2022/0265816 A1
(43) Pub. Date: Aug. 25, 2022

(54) USE OF THE AGENT FOR INDUCTION OF SPECIFIC IMMUNITY AGAINST SEVERE ACUTE RESPIRATORY SYNDROME VIRUS SARS-COV-2 FOR REVACCINATION OF POPULATION (VARIANTS)

(71) Applicant: Federal State Budgetary Institution "National Research Centre for Epidemiology and Microbiology name, Moscow (RU)

(72) Inventors: Olga Vadimovna ZUBKOVA, Khimki (RU); Tatiana Andreevna OZHAROVSKAIA, Korolev (RU); Inna Vadimovna DOLZHIKOVA, Lobnya (RU); Olga POPOVA, Ufa (RU); Dmitrii Viktorovich SHCHEBLIAKOV, Moscow (RU); Daria Mikhailovna GROUSOVA, Moscow (RU); Alina Shahmirovna DZHARULLAEVA, Moscow (RU); Amir Ildarovich TUKHVATULIN, Moscow (RU); Natalia Mikhailovna TUKHVATULINA, Moscow (RU); Dmitrii Nikolaevich SHCHERBININ, Moskovskaya oblasr (RU); Ilias Bulatovich ESMAGAMBETOV, Dmitrov (RU); Elizaveta Alexsandrovna TOKARSKAYA, Moscow (RU); Andrei Gennadevich BOTIKOV, Moscow (RU); Alina Sergeevna EROXOVA, Bryansk (RU); Fatima Magometovna IZHAEVA, Karahaevo-Cherkesskaya Respublika (RU); Natalya Anatolevna NIKITENKO, Moscow (RU); Nadezhda Leonidovna LUBENETS, Moskovskaya oblast (RU); Aleksandr Sergeevich SEMIKHIN, Moscow (RU); Vladimir Aleksandrovich CHERNETSOV, Moscow (RU); Evgenii Vladimirovich KRIUKOV, Moscow (RU); Vladimir Fedorovich BABIRA, Moskovskaya oblast (RU); Dmitrii Anatolevich KUTAEV, Moskovskaya oblast (RU); Svetlana Iakovlevna LOGINOVA, Moskovskaya oblast (RU); Boris Savelievich NARODITSKY, Moscow (RU); Denis Yuryevich LOGUNOV, Moscow (RU); Aleksandr Leonidovich GINTSBURG, Moscow (RU)

(21) Appl. No.: 17/722,227

(22) Filed: Apr. 15, 2022

Related U.S. Application Data
(63) Continuation of application No. PCT/RU2022/000046, filed on Feb. 18, 2022.

(30) Foreign Application Priority Data
Feb. 21, 2021 (RU) 2021104437

(51) Int. Cl. A61K 39/215; A61P 31/14; C12N 15/86; C12N 7/00

(52) U.S. Cl. CPC A61K 39/215; A61P 31/14; C12N 15/86; C12N 7/00

(57) ABSTRACT

The disclosed invention relates to safe and efficacious methods of extending postvaccinal immunity against severe acute respiratory syndrome virus SARS-CoV-2 and revaccinating a population against diseases caused by SARS-CoV-2. The disclosed methods include administration of an agent to a person or population. The agent may include a first component comprising an expression vector based on a genome of recombinant strain of (i) human adenovirus serotype 26 with the E1 and E3 sites deleted from the genome and the site ORF6-Ad26 substituted for ORF6-Ad5 with an integrated expression cassette selected from SEQ ID NO:1, SEQ ID NO:2, or SEQ ID NO:3, or (ii) a simian adenovirus serotype 25 with the E1 and E3 sites deleted from the genome with an integrated expression cassette selected from SEQ ID NO:4, SEQ ID NO:2, or SEQ ID NO:3. The first component of the agent is combined or replaced with a second component in the form of an expression vector based on a genome of recombinant strain of human adenovirus serotype 5 with the E1 and E3 sites deleted from the genome with an integrated expression cassette selected from SEQ ID NO:1, SEQ ID NO:2, or SEQ ID NO:3. In addition to the above, the method may include administering an agent as disclosed herein, wherein the first component or the second component has the form of an expression vector based on genome of recombinant strain of human adenovirus serotype 5 with E1 and E3 sites deleted from the genome with the integrated expression cassette selected from SEQ ID NO:1, SEQ ID NO:2, or SEQ ID NO:3.

### CROSS-REFERENCE TO RELATED APPLICATIONS

The present application is a continuation application of International Application No. PCT/RU2022/000046, filed Feb. 18, 2022, which claims priority to Russian Patent Application No. 2021104437, filed on Feb. 21, 2021, the contents of both applications are hereby incorporated by reference in their entirety.

### INCORPORATION BY REFERENCE-SEQUENCE LISTING

This application includes an electronically submitted sequence listing in .txt format. The .txt file contains a sequence listing entitled “110620_00626 SequenceListing.txt” which was created on Apr. 15, 2022 and is 163,230 bytes in size. The sequence listing contained in this .txt file is part of the specification and is hereby incorporated by reference herein in its entirety.

### FIELD OF THE INVENTION

The group of invention relates to biotechnology, immunology and virology. The use of the agents for revaccination of population against the diseases caused by severe acute respiratory syndrome virus SARS-CoV-2.

### BACKGROUND OF THE INVENTION

Coronaviruses is a large virus family, which cause a wide spectrum of diseases in humans and animals. At the end of 2019 the world faced with a novel zoonotic beta-coronavirus SARS-CoV-2, which cause the outbreak of coronavirus infection (COVID-19) in Wuhan (People's Republic of China (PRC)). On Mar. 11, 2020, the World Health Organization described the spread of the disease in the world as a pandemic. As of Feb. 1, 2021, more than 100 million cases of COVID-19 illnesses were recorded, and more than 2 million people died.

The most common symptoms of COVID-19 include fever, dry cough, dyspnea, and fatigue. Sore throat, pain in joints, running nose, and headache occur more rarely. The illness may have mild or severe course. Advanced age and the presence of chronic diseases are the risk factors.

After the illness both cell-mediated and antibody-mediated immune responses are formed. CD8+ and CD4+ T cells specific to SARS-CoV-2 are found in 70% and 100% of COVID-19 convalescents, respectively. S protein of SARS-CoV-2 is the main target for T cells. In addition, T cells specific to M and N coronavirus proteins are found and less numerous T cells specific to nsp3, nsp4, ORF3a and ORF8 of SARS-CoV-2. Immune response is polarized towards Th1 (Grifoni et al. Targets of T Cell Responses to SARS-CoV-2 Coronavirus in Humans with COVID-19 Disease and Unexposed Individuals. Cell. 2020 Jun. 25; 181(7): 1489-1501.e15).

Antibody immune response is mediated by the antibodies targeted primarily to coronavirus surface S protein. It is shown that RBD of S glycoprotein, which is responsible for binding with ACE-2 receptor on human cells is the main target for virus-neutralizing antibodies. Kinetics of the antibody-mediated immune response against SARS-CoV-2 is characterized by sustainable seroconversion (IgM and IgG) within 7 to 14 days after the symptom appearance. IgG titers increase during the first 3 weeks and begin decreasing to week 8 (Adams ER, Ainsworth M, Anand R. Antibody testing for COVID-19: a report from the National COVID Scientific Advisory Panel. medRxiv. 2020). Furthermore, it was demonstrated that IgG titers correlate with the severity of the disease. (Gregory A Poland et al. SARS-CoV-2 immunity: review and applications to phase 3 vaccine candidates. Lancet. 2020 14-20 Nov.; 396(10262): 1595-1606).

Scientific data accumulated so far are indicative of rather short-term natural immunity to COVID-19, which is formed in a subject who experienced the disease (https://www.cdc.gov/coronavirus/2019-ncov/vaccines/facts.html). This is evident from the observed tendencies of decreasing antibody levels and the cases of coronavirus reinfection (Akiko Iwasaki. What reinfections mean for COVID-19. Lancet Infect Dis. 2021 Jan; 21(1): 3-5.)

Vaccination is the most effective method of infectious disease prevention. By present several COVID-19 vaccines have been developed, which are based on various coronavirus antigens.
1) Vaccines containing a whole virus as an antigen.
   Four candidate inactivated vaccines developed in China are known. At present 3 vaccines (Sinovac in collaboration with National Institute for Prevention And Control of Infectious Diseases; Sinopharm in collaboration with Wuhan Institute of Biological Products and Wuhan Institute of Virology of Chinese Academy of Sciences; Sinopharm in collaboration with Beijing Institute of Biological Products and Institute of Control And Prevention of Viral Diseases) are studied in phase III of clinical programs, and one vaccine (Institute of medical biology, Chinese Academy of Medical sciences) is studied in phase I/II of clinical program.
   (Zhang Y et al. Safety, tolerability, and immunogenicity of an inactivated SARS-CoV-2 vaccine in healthy adults aged 18-59 years: a randomised, double-blind, placebo-controlled, phase 1/2 clinical trial. Lancet Infect Dis. 2021 Feb;21(2):181-192; Xia S et al. Effect of an Inactivated Vaccine Against SARS-CoV-2 on Safety and Immunogenicity Outcomes: Interim Analysis of 2 Randomized Clinical Trials. JAMA. 2020 Sep. 8;324(10):951-960. Xia S et al Safety and immunogenicity of an inactivated SARS-CoV-2 vaccine, BBIBP-CorV: a randomised, double-blind, placebo-controlled, phase 1/2 trial. Lancet Infect Dis. 2021 Jan;21(1):39-51. doi: 10.1016/S1473-3099(20)30831-8. Epub 2020 Oct. 15. PMID: 33069281; PMCID: PMC7561304. Che Y et al. Randomized, double-blinded and placebo-controlled phase II trial of an inactivated SARS-CoV-2 vaccine in healthy adults. Clin Infect Dis. 2020 Nov. 9).
   2) Vaccines containing a full-length S protein as an antigen.
   Three vaccines based on adenoviruses of various serotypes, which express the gene of full-length S protein of SARS-CoV-2 are known. CanSino Biological Inc. [symbol] Beijing Institute of Biotechnology have developed the vaccine based on human adenoviruse serotype 5; Oxford University, AstraZeneca— the vaccine based on chimpanzee adenovirus; FGBU N.F.Gamaleya National Research Center For Epidemiology And Microbiology, Ministry of Health of Russia—the vaccine based on human adenoviruses serotype 26 and serotype 5. In addition DNA vaccine is known, which contains the gene of full-length S protein of SARS-CoV-2, and was developed by Inovio Pharmaceuticals in collaboration with International Vaccine Institute.
   (Zhu F et al. Safety, tolerability, and immunogenicity of a recombinant adenovirus type-5 vectored COVID-19 vaccine: a dose-escalation, open-label, non-randomised, first-in-human trial. Lancet. 2020 Jun. 13;395(10240):1845-1854. van Doremalen N et al. ChAdOx1 nCoV-19 vaccine prevents SARS-CoV-2 pneumonia in rhesus macaques. Nature. 2020 October; 586(7830):578-582. Logunov DY et al. Safety and immunogenicity of an rAd26 and rAd5 vector-based heterologous prime-boost COVID-19 vaccine in two formulations: two open, non-randomised phase 1/2 studies from Russia. Lancet. 2020 Sep 26;396(10255):887-897).
   3) Vaccines, in which full-length S protein with two proline substitutions (K986P [symbol] V987P) serves as an antigen.
   Two vaccines are known, which are based on lipid nanoparticles containing mRNA encoding S protein of SARS-CoV-2 with proline substitutions (Moderna in collaboration with National Institute of Allergy And Infectious Diseases; BioNTech in collaboration with Fosun Pharma and Pfizer). In addition, protein subunit vaccine developed by Novavax is known; in that vaccine full-length S protein of SARS-CoV-2 with two proline substitutions (K986P [symbol] V987P) and three mutations in the furin cleavage site (R682Q, R683Q [symbol] R685Q). Another vaccine based on human adenoviruse serotype 26 expressing full-length S protein of SARS-CoV-2 with two proline substitutions (K986P [symbol] V987P) and two mutations in the furin cleavage site (R682S [symbol] R685G) has been developed by Janssen Pharmaceutical Companies.
   (L. Baden et al. Efficacy and Safety of the mRNA-1273 SARS-CoV-2 Vaccine. N Engl J Med. 2020 Dec. 30; L. Jackson An mRNA Vaccine against SARS-CoV-2—Preliminary Report. N Engl J Med. 2020 Nov. 12;383(20):1920-1931. Keech C et al. Phase 1-2 Trial of a SARS-CoV-2 Recombinant Spike Protein Nanoparticle Vaccine. N Engl J Med. 2020 Dec. 10; Tostanoski L et al. Ad26 vaccine protects against SARS-CoV-2 severe clinical disease in hamsters. Nat Med. 2020 Nov;26(11):1694-1700. doi: 10.1038/s41591-020-1070-6. Epub 2020 Sep. 3).
   4) Vaccines containing RBD of S protein as an antigen
   Protein subunit vaccine containing RBD-dimer (residues 319-537 as a tandem repeat) has been developed by Anhui Zhifei Longcom Biopharmaceutical in collaboration with Institute of Microbiology of Chinese Academy of Sciences. In addition, the vaccine based on lipid nanoparticles containing mRNA encoding RBD-trimer (trimerized by addition foldon domain from of T4 fibritin is known (BioNTech in collaboration with Fosun Pharma and Pfizer) (Mulligan M. et al. Phase I/II study of COVID-19 RNA vaccine BNT162b1 in adults. Nature. 2020 October;586(7830):589-593. Dai L. A Universal Design of Betacoronavirus Vaccines against COVID-19, MERS, and SARS. Cell. 2020 Aug. 6;182(3):722-733.e11. Dai Let al. Viral targets for vaccines against COVID-19. Nat Rev Immunol. 2021 February;21(2):73-82).

At present 8 vaccines for COVID-19 prevention are authorized in the world. The clinical study results showed that immunization with these vaccines results in development of both antibody-mediated and cell-mediated immune response against SARS-CoV-2. However, how durable post-vaccinal protective immunity is provided by each type of vaccines is not yet established. In the opinion of experts, it may show interindividual variability and according to various estimates lasts from 1 to 2 years. Such duration determines the need for development of specific agents for COVID prophylaxis to be used in revaccination of humans.

However, the selection of revaccination agents is a challenging task.

In the course of developing the agents for specific prophylaxis intended for revaccination one should keep in mind the effects arising in humans from booster immunization, which have considerable impact on overall structure of antiinfective immunity including protective properties thereof

It is known that the use of vaccines comprising numerous antigens (for instance, inactivated vaccines) leads to formation of immune response against each antigen. However, in this event the immunity level in respect to given antigen is lower compared to vaccines comprising merely this one antigen (effect of immune response dilution). Furthermore, some antigens in the pathogen structure may be non-protective, and formation of T- and B-cell clones in response against such antigens will not contribute to overall protectivity of immunity interfering with formation the cell clones, which are important for protection.

Based on the above one can conclude that vaccines comprising one or more proteins with pronounced protective properties are more promising for revaccination. In this event revaccination will be associated with additional stimulation (boosting) and equally important focusing of the immune response on antigenic determinants of the pathogen, which are most important for the human protection irrespectively of initial immunization.

No agents for revaccination against coronavirus infection are known from the state of the art.

Technical solution disclosed in RF patent No 2731342 (published on Jan. 1,2020) was chosen by the authors of the claimed invention as a prototype. The variants of agent for induction of specific immunity against severe acute respiratory syndrome virus SARS-CoV-2 are known from this patent.

containing component 1, which is an agent in the form of expression vector based on genome of recombinant strain of human adenovirus serotype 26 with E1 and E3 sites deleted from the genome, and the site ORF6-Ad26 is substituted for ORF6-Ad5 with integrated expression cassette selected from SEQ ID NO:1, SEQ ID NO:2, SEQ ID NO:3, and also containing a component 2, which is an agent in the form of expression vector based on genome of recombinant strain of human adenovirus serotype 5 with E1 and E3 sites deleted from the genome with integrated expression cassette selected from SEQ ID NO:1, SEQ ID NO:2, SEQ ID NO:3.

containing a component 1, which is an agent in the form of expression vector based on genome of recombinant strain of human adenovirus serotype 26 with E1 and E3 sites deleted from the genome, and the site ORF6-Ad26 is substituted for ORF6-Ad5 with integrated expression cassette selected from SEQ ID NO:1, SEQ ID NO:2, SEQ ID NO:3, and also containing a component 2, which is an agent in the form of expression vector based on genome of recombinant strain of simian adenovirus serotype 25 with E1 and E3 sites deleted from the genome with integrated expression cassette selected from SEQ ID NO:4, SEQ ID NO:2, SEQ ID NO:3.

containing a component 1, which is an agent in the form of expression vector based on genome of recombinant strain of simian adenovirus serotype 25 with E1 and E3 sites deleted from the genome with integrated expression cassette selected from SEQ ID NO:4, SEQ ID NO:2, SEQ ID NO:3, and also containing a component 2, which is an agent in the form of expression vector based on genome of recombinant strain of human adenovirus serotype 5 with E1 and E3 sites deleted from the genome with the integrated expression cassette selected from SEQ ID NO:1, SEQ ID NO:2, SEQ ID NO:3.

In addition, in this patent the use of indicated variants of agent for induction of specific immunity against severe acute respiratory syndrome virus SARS-CoV-2 is disclosed, including administration of component 1 and component 2 in effective amounts sequentially at a time interval of at least one week.

The disadvantage of this agent is that the use thereof for prolongation of postvaccinal immunity is not described.

Therefore, background of the invention shows a need for developing an agent, which can be used for revaccination against the diseases caused by SARS-CoV-2 virus

### DISCLOSURE OF THE INVENTION

Technical problem of the claimed group of invention is development of the agents providing prolongation of postvaccinal immunity against SARS-CoV-2 virus.

Technical result is the creation of safe and efficacious agent providing prolongation of postvaccinal immunity against SARS-CoV-2 virus.

Said technical result is achieved through disclosure of using an agent containing component 1, which is an agent in the form of expression vector based on genome of recombinant strain of human adenovirus serotype 26 with E1 and E3 sites deleted from the genome, and the site ORF6-Ad26 is substituted for ORF6-Ad5 with integrated expression cassette selected from SEQ ID NO:1, SEQ ID NO:2, SEQ ID NO:3, and/or containing a component 2, which is an agent in the form of expression vector based on genome of recombinant strain of human adenovirus serotype 5 with E1 and E3 sites deleted from the genome with integrated expression cassette selected from SEQ ID NO:1, SEQ ID NO:2, SEQ ID NO:3 for revaccination against the diseases caused by severe acute respiratory syndrome virus SARS-CoV-2.

In addition, the use of the agent is disclosed, said agent containing a component 1, which is an agent in the form of expression vector based on genome of recombinant strain of human adenovirus serotype 26 with E1 and E3 sites deleted from the genome, and the site ORF6-Ad26 is substituted for ORF6-Ad5 with integrated expression cassette selected from SEQ ID NO:1, SEQ ID NO:2, SEQ ID NO:3, and also containing a component 2, which is an agent in the form of expression vector based on genome of recombinant strain of simian adenovirus serotype 25 with E1 and E3 sites deleted from the genome with integrated expression cassette selected from SEQ ID NO:4, SEQ ID NO:2, SEQ ID NO:3, or containing only component 2 for revaccination against the diseases caused by severe acute respiratory syndrome virus SARS-CoV-2.

In addition, the use of another agent is disclosed, said agent containing a component 1, which is an agent in the form of expression vector based on genome of recombinant strain of simian adenovirus serotype 25 with E1 and E3 sites deleted from the genome with integrated expression cassette selected from SEQ ID NO:4, SEQ ID NO:2, SEQ ID NO:3, and also containing a component 2, which is an agent in the form of expression vector based on genome of recombinant strain of human adenovirus serotype 5 with E1 and E3 sites deleted from the genome with the integrated expression cassette selected from SEQ ID NO:1, SEQ ID NO:2, SEQ ID NO:3 for revaccination against the diseases caused by severe acute respiratory syndrome virus SARS-CoV-2.

Said agent is used in liquid or lyophilized form.

What is more, buffer for the liquid form contains, % by weight:

| | |
|---|---|
| tris | 0.1831 to 0.3432 |
| sodium chloride | 0.3313 to 0.6212 |
| saccharose | 3.7821 to 7.0915 |
| magnesium chloride hexahydrate | 0.0154 to 0.0289 |
| EDTA | 0.0029 to 0.0054 |
| polysorbate-80 | 0.0378 to 0.0709 |
| ethanol 95% | 0.0004 to 0.0007 |
| water | balance. |

In addition, reconstituted lyophilized agent contains buffer composed of, % by weight:

| | |
|---|---|
| tris | 0.0180 to 0.0338 |
| sodium chloride | 0.1044 to 0.1957 |
| saccharose | 5.4688 to 10.2539 |
| magnesium chloride hexahydrate | 0.0015 to 0.0028 |
| EDTA | 0.0003 to 0.0005 |
| polysorbate-80 | 0.0037 to 0.0070 |
| water | balance. |

In addition, during the use a component 1 and a component 2 are in separate containers.

### Short Description of the Figures

FIG. 1

Illustrates the results of assessment of immunization efficacy in volunteers using a liquid form of the developed agent according to variant 1 through assessment of the percentage of proliferating CD8+ lymphocytes restimulated by S antigen of SARS-CoV-2.

Y-axis—An amount of proliferating cells, %.

X-axis—Days.

●—indicates individual values of each volunteer on Day 0.

[symbol] —indicates individual values of each volunteer on Day 14.

Δ—indicates individual values of each volunteer on Day 28.

The median value is shown as a black line for each data set. Statistically significant difference between the values obtained on days 0. 14 and 28 is shown by bracket and symbols *, p<0.05; **, p<0.01; ****, p<0.001, using Mann-Whitney test.

FIG. 2

Illustrates the results of assessment of immunization efficacy in volunteers using a liquid form of the developed agent according to variant 1 through assessment of the percentage of proliferating CD4+ lymphocytes restimulated by S antigen of SARS-CoV-2.

Y-axis—An amount of proliferating cells, %.

X-axis—Days.

● indicates individual values of each volunteer on Day 0.

[symbol] —indicates individual values of each volunteer on Day 14.

Δ—indicates individual values of each volunteer on Day 28.

The median value is shown as a black line for each data set. Statistically significant difference between the values obtained on days 0. 14 and 28 is shown by bracket and symbols *, p<0.05; **, p<0.01; ****, p<0.001, using Mann-Whitney test.

FIG. 3

Illustrates the results of assessment of immunization efficacy in volunteers using a lyophilized form of the developed agent according to variant 1 through assessment of the percentage of proliferating CD8+ lymphocytes restimulated by S antigen of SARS-CoV-2.

Y-axis—An amount of proliferating cells, %.

X-axis—Days.

●—indicates individual values of each volunteer on Day 0.

[symbol] —indicates individual values of each volunteer on Day 14.

Δ—indicates individual values of each volunteer on Day 28.

The median value is shown as a black line for each data set. Statistically significant difference between the values obtained on days 0. 14 and 28 is shown by bracket and symbols *, p<0.05; **, p<0.01; ****, p<0.001, using Mann-Whitney test.

FIG. 4

Illustrates the results of assessment of immunization efficacy in volunteers using a lyophilized form of the developed agent according to variant 1 through assessment of the percentage of proliferating CD4+ lymphocytes restimulated by S antigen of SARS-CoV-2.

Y-axis—An amount of proliferating cells, %.

X-axis—Days.

●—indicates individual values of each volunteer on Day 0.

[symbol] —indicates individual values of each volunteer on Day 14.

Δ—indicates individual values of each volunteer on Day 28.

The median value is shown as a black line for each data set. Statistically significant difference between the values obtained on days 0. 14 and 28 is shown by bracket and symbols *, p<0.05; **, p<0.01; ****, p<0.001, using Mann-Whitney test.

FIG. 5

Illustrates the fold increase in IFNγ concentration in the culture medium of peripheral blood mononuclear cells of the volunteers, who were immunized with the liquid form of the developed agent according to variant 1, after restimulation with S antigen of SARS-CoV-2 before immunization (Day 0) and on Days 14 and 28 of the study.

Y-axis—Fold increase in interferon-gamma concentration

X-axis—Days.

●—indicates individual values of each volunteer on Day 0.

[symbol] —indicates individual values of each volunteer on Day 14.

Δ—indicates individual values of each volunteer on Day 28.

The median value is shown as a black line for each data set. Statistically significant difference between the values obtained on days 0. 14 and 28 is shown by bracket and symbols *, p<0.05; **, p<0.01; ****, p<0.001, using Mann-Whitney test.

FIG. 6

Illustrates the fold increase in IFNγ concentration in the culture medium of peripheral blood mononuclear cells of the volunteers, who were immunized with the lyophilized form of the developed agent according to variant 1, after restimulation with S antigen of SARS-CoV-2 before immunization (Day 0) and on Days 14 and 28 of the study.

Y-axis—Fold increase in interferon-gamma concentration

X-axis—Days.

●—indicates individual values of each volunteer on Day 0. [symbol] —indicates individual values of each volunteer on Day 14.

Δ—indicates individual values of each volunteer on Day 28.

The dots show individual values of each volunteer who participated in the study. The median value is shown as a black line for each data set. Statistically significant difference between the values obtained on days 0. 14 and 28 is shown by bracket and symbols *, p<0.05; **, p<0.01; ****, p<0.001, using Mann-Whitney test.

FIG. 7

Illustrates the results of assessment of the antibody-mediated immune response against the antigen of SARS-CoV2 in the volunteers, who were immunized with the liquid form of the developed agent according to variant 1.

Y-axis—Titer of IgG against RBD of S glycoprotein of SARS-CoV-2.

X-axis—Days.

[symbol] —values of each volunteer.

FIG. 8

Illustrates the results of assessment of the antibody-mediated immune response against the antigen of SARS-CoV2 in the volunteers, who were immunized with the lyophilized form of the developed agent according to variant 1.

Y-axis—Titer of IgG against RBD of S glycoprotein of SARS-CoV-2.

X-axis—Days.

[symbol] —values of each volunteer.

The first stage in the development of the agent for inducing specific immunity against severe acute respiratory syndrome virus SARS-CoV-2 was the selection of a vaccine antigen. As a part of this work, the literature search was performed which demonstrated that the coronavirus S protein was the most promising antigen for creating a candidate vaccine. This type I transmembrane glycoprotein is responsible for virus particles binding, fusion and entry into the cells. As was shown, it induces the production of neutralizing antibodies (Liang M et al, SARS patients-derived human recombinant antibodies to S and M proteins efficiently neutralize SARS-coronavirus infectivity. Biomed Environ Sci. 2005 December;18(6):363-74).

The authors developed various variants of the expression cassettes to achieve the most effective induction of immune response against S protein of SARS-CoV-2.

### Implementation of the Invention

Expression cassette SEQ ID NO:1 comprises CMV promoter, gene encoding S protein of SARS-CoV-2 and polyadenylation signal. CMV promoter is a promoter of early cytomegalovirus genes, which provides constitutive expression in numerous cell types. However, the strength of expression of the target gene controlled by CMV promoter varies depending on the cell type. In addition, it was shown that the level of transgene expression controlled by CMV promoter decreases with longer cell cultivation time because of inhibition of the gene expression related to DNA methylation [Wang W., Jia YL., Li YC., Jing CQ., Guo X., Shang XF., Zhao CP., Wang T Y. Impact of different promoters, promoter mutation, and an enhancer on recombinant protein expression in CHO cells. //Scientific Reports—2017.—Vol. 8.—P. 10416].

Expression cassette SEQ ID NO:2 comprises CAG promoters, gene encoding S protein of SARS-CoV-2 and polyadenylation signal. CAG-promoter is a synthetic promoter, which switch on the early enhancer of CMV promoter, chicken [3-actin promoter and chimeric intron (chicken β-actin and rabbit β-globin). The experiments show that transcriptional activity of CAG promoter is higher compared to CMV promotor. [Yang C. Q., Li X. Y., Li Q., Fu S. L., Li H., Guo Z. K., Lin J. T., Zhao S. T. Evaluation of three different promoters driving gene expression in developing chicken embryo by using in vivo electroporation. II Genet. Mol. Res.—2014. —Vol. 13. —P. 1270-1277].

Expression cassette SEQ ID NO:3 comprises EF1 promoters, gene encoding S protein of SARS-CoV-2 and polyadenylation signal. EF1 promoter is a promoter of human eukaryotic translation elongation factor 1β (EF-1α). The promoter is constitutively active in the wide range of cell types [PMID: 28557288. The EF-1α promoter maintains high-level transgene expression from episomal vectors in transfected CHO-K1 cells]. Gene EF-α encodes the elongation factor 1α, which is one of the most common proteins in eukaryotic cells and is expressed almost in all cell types of the mammals. This EF-1α is often active in the cells where the viral promoters are not able to express the controlled genes, and in the cells, where the viral promoters are gradually fade away.

Expression cassette SEQ ID NO:4 comprises CMV promoter, gene encoding S protein of SARS-CoV-2, and polyadenylation signal.

Adenovirus-based vector system was selected for effective delivery of the gene encoding S protein of SARS-CoV-2 coronavirus into the human body. Adenoviral vectors provide a number of advantages: they cannot reproduce in the human cells, enter both dividing and nondividing cells, are able to induce cell-mediated and antibody-mediated immune response, and provide high level of the target antigen expression.

The authors developed the variants of the agent containing two components, which are based on different adenovirus serotypes. In this event the immune response against the vector part of adenovirus, which can develop after administration of the first component of the agent, in future is not boosted and does not affect the generation of antigen-specific immune response against vaccine antigen.

Furthermore, the developed agents expand armamentarium of the agents for inducing the immune response against SARS-CoV-2 coronavirus, and this will provide overcoming the difficulties arisen from the presence of preexisting immune response against some adenovirus serotypes in some part of population.

Thus, the efforts resulted in development of the following agent variants.
1. The agent for revaccination against the diseases caused by severe acute respiratory syndrome virus SARS-CoV-2, containing component 1, which is an agent in the form of expression vector based on genome of recombinant strain of human adenovirus serotype 26 with E1 and E3 sites deleted from the genome, and the site ORF6-Ad26 is substituted for ORF6-Ad5 with integrated expression cassette selected from SEQ ID NO:1, SEQ ID NO:2, SEQ ID NO:3, and/or containing a component 2, which is an agent in the form of expression vector based on genome of recombinant strain of human adenovirus serotype 5 with E1 and E3 sites deleted from the genome with integrated expression cassette selected from SEQ ID NO:1, SEQ ID NO:2, SEQ ID NO:3
2. The agent for revaccination against the diseases caused by severe acute respiratory syndrome virus SARS-CoV-2, containing containing a component 1, which is an agent in the form of expression vector based on genome of recombinant strain of human adenovirus serotype 26 with E1 and E3 sites deleted from the genome, and the site ORF6-Ad26 is substituted for ORF6-Ad5 with integrated expression cassette selected from SEQ ID NO:1, SEQ ID NO:2, SEQ ID NO:3, and also containing a component 2, which is an agent in the form of expression vector based on genome of recombinant strain of simian adenovirus serotype 25 with E1 and E3 sites deleted from the genome with integrated expression cassette selected from SEQ ID NO:4, SEQ ID NO:2, SEQ ID NO:3, or containing only component 2.
3. The agent for revaccination against the diseases caused by severe acute respiratory syndrome virus SARS-CoV-2, containing a component 1, which is an agent in the form of expression vector based on genome of recombinant strain of simian adenovirus serotype 25 with E1 and E3 sites deleted from the genome with integrated expression cassette selected from SEQ ID NO:4, SEQ ID NO:2, SEQ ID NO:3, and also containing a component 2, which is an agent in the form of expression vector based on genome of recombinant strain of human adenovirus serotype 5 with E1 and E3 sites deleted from the genome with the integrated expression cassette selected from SEQ ID NO:1, SEQ ID NO:2, SEQ ID NO:3.

### EXAMPLE 1

Obtaining of expression vector containing genome of recombinant strain of human adenovirus serotype 26.

At Stage 1 the authors developed the design of plasmid construct pAd26-Ends, which carries two sites homologous to genome of human adenovirus serotype 26 (two homology arms) and ampicillin resistance gene. One homology arm is a beginning of human adenovirus serotype 26 (from the left inverted terminal repeat to E1 site) and the viral genome sequence including pIX protein. The second homology arm contains the nucleotide sequence from ORF3 of E4 site to the end of genome. pAd26-Ends construct was synthesized by ZAO “Eurogene” (Moscow).

DNA of human adenovirus serotype 26 isolated from the virions was mixed with pAd26-Ends construct. Homologous recombination between pAd26-Ends and viral DNA resulted in plasmid pAd26-d1E1, which carries the genome of human adenovirus serotype 26 with E1 site deleted.

Then in the obtained plasmid pAd26-d1E1 the sequence containing open reading frame 6 (ORF6-Ad26) was replaced with analogous sequence from the human adenovirus serotype 5 using the conventional cloning methods, to enable effective replication of human adenovirus serotype 26 in the cell culture HEK293. This resulted in plasmid pAd26-d1E1-ORF6-Ad5.

Then E3 site of the adenovirus genome (about 3321 b.p. between pIII gene and U-exon) was deleted from the constructed plasmid pAd26-d1E1-ORF6-Ad5 using conventional genetic engineering methods to increase the vector packing capacity. This resulted in recombinant vector pAd26-only-null based on genome of recombinant strain of human adenovirus serotype 26 containing open reading frame ORF6 of human adenovirus serotype 5 and deleted E1 and E3 sites of the genome. SEQ ID NO:5 was used as a maternal sequence of human adenovirus serotype 26.

In addition, the authors developed several designs of the expression cassette:

expression cassette SEQ ID NO:1 comprises CMV promoter, the gene encoding S protein of SARS-CoV-2, and polyadenylation signal;

expression cassette SEQ ID NO:2 comprises CAG promoter, the gene encoding S protein of SARS-CoV-2 and polyadenylation signal;

expression cassette SEQ ID NO:3 comprises EF1 promoter, the gene encoding S protein of SARS-CoV-2, and polyadenylation signal.

On the basis of plasmid construct pAd26-Ends the constructs pArms-26-CMV-S-CoV2, pArms-26-CAG-S-CoV2, pArms-26-EF1-S-CoV2, containing expression cassettes SEQ ID NO:1, SEQ ID NO:2 or SEQ ID NO:3, respectively, and also bearing homology arms of the genome of human adenovirus serotype 26 were obtained using the genetic engineering methods. Then the constructs pArms-26-CMV-S-CoV2, pArms-26-CAG-S-CoV2, pArms-26-EF1-S-CoV2 were linearized at the unique hydrolysis site between the homology arms, each plasmid was mixed with recombinant vector pAd26-only-null. Homologous recombination resulted in plasmids pAd26-only-CMV-S-CoV2, pAd26-only-CAG-S-CoV2, pAd26-only-EF1-S-CoV2, carrying the genome of recombinant strain of human adenovirus serotype 26 containing open reading frame ORF6 of human adenovirus serotype 5 and deleted E1 and E3 sites of the genome, with expression cassette SEQ ID NO:1, SEQ ID NO:2 or SEQ ID NO:3, respectively.

At Stage 4 the plasmids pAd26-only-CMV-S-CoV2, pAd26-only-CAG-S-CoV2, pAd26-only-EF1-S-CoV2 were hydrolyzed with specific restriction endonucleases to remove the vector part. The obtained DNA products were used for transfection of the cell culture HEK293.

Thus, the expression vector was obtained, containing the genome of recombinant strain of human adenovirus serotype 26 with E1 and E3 sites deleted from the genome, and the site ORF6-Ad26 is substituted for ORF6-Ad5 with integrated expression cassette selected from SEQ ID NO:1, SEQ ID NO:2, SEQ ID NO:3.

### EXAMPLE 2

Obtaining of immunobiological agent in the form of expression vector based on genome of recombinant strain of human adenovirus serotype 26, in which E1 and E3 sites are deleted from the genome, and the site ORF6-Ad26 is substituted for ORF6-Ad5 with integrated expression cassette selected from SEQ ID NO:1, SEQ ID NO:2, SEQ ID NO:3.

At this stage of the work the expression vectors obtained in Example 1 were purified using anion exchange and exclusion chromatography. Resultant suspension contained adenovirus particles in the buffer for liquid form of the agent or in the buffer for lyophilized form of the agent.

Thus, the following immunobiological agents based on genome of recombinant strain of human adenovirus serotype 26, with E1 and E3 sites deleted from the genome, and the site ORF6-Ad26 substituted for ORF6-Ad5 were obtained:

1. Immunobiological agent based on genome of recombinant strain of human adenovirus serotype 26, in which E1 and E3 sites are deleted from the genome, and the site ORF6-Ad26 is substituted for ORF6-Ad5 with expression cassette containing CMV promoter, the gene encoding S protein of SARS-CoV-2 and polyadenylation signal, SEQ ID NO:1 (Ad26-CMV-S-CoV2) in the buffer for liquid form of the agent.

2. Immunobiological agent based on genome of recombinant strain of human adenovirus serotype 26, in which E1 and E3 sites are deleted from the genome, and the site ORF6-Ad26 is substituted for ORF6-Ad5 with expression cassette containing CMV promoter, the gene encoding S protein of SARS-CoV-2 and polyadenylation signal, SEQ ID NO:1 (Ad26-CMV-S-CoV2) in the buffer for lyophilized form of the agent.

3. Immunobiological agent based on genome of recombinant strain of human adenovirus serotype 26, in which E1 and E3 sites are deleted from the genome, and the site ORF6-Ad26 is substituted for ORF6-Ad5 with expression cassette containing CAG promoter, the gene encoding S protein of SARS-CoV-2 and polyadenylation signal, SEQ ID NO:2 (Ad26-CAG-S-CoV2) in the buffer for liquid form of the agent.

4. Immunobiological agent based on genome of recombinant strain of human adenovirus serotype 26, in which E1 and E3 sites are deleted from the genome and the site ORF6-Ad26 is substituted for ORF6-Ad5 with expression cassette containing CAG promoter, the gene encoding S protein of SARS-CoV-2 and polyadenylation signal, SEQ ID NO:2 (Ad26-CAG-S-CoV2) in the buffer for lyophilized form of the agent.

5. Immunobiological agent based on genome of recombinant strain of human adenovirus serotype 26, in which E1 and E3 sites are deleted from the genome and the site ORF6-Ad26 is substituted for ORF6-Ad5 with expression cassette containing EF1 promoter, the gene encoding S protein of SARS-CoV-2 and polyadenylation signal, SEQ ID NO:3 (Ad26-EF1-S-CoV2) in the buffer for liquid form of the agent.

6. Immunobiological agent based on genome of recombinant strain of human adenovirus serotype 26, in which E1 and E3 sites are deleted from the genome, and the site ORF6-Ad26 is substituted for ORF6-Ad5 with expression cassette containing EF1 promoter, the gene encoding S protein of SARS-CoV-2 and polyadenylation signal, SEQ ID NO:3 (Ad26-EF1-S-CoV2) in the buffer for lyophilized form of the agent.

Each of provided immunobiological agents is a component 1 in variant 1 and in variant 2 of the developed agent.

### EXAMPLE 3

Obtaining of expression vector containing the genome of recombinant strain of simian adenovirus serotype 25.

At Stage 1 the design of plasmid construct pSim25-Ends carrying two sites homologous to the genome of simian adenovirus serotype 25 (two homology arms) was developed. One homology arm is a beginning of simian adenovirus serotype 25 (from the left inverted terminal repeat to E1 site) and the sequence from the end of E1 site to pIVa2 protein. The second homology arm contains the end nucleotide sequence of adenovirus genome including right inverted terminal repeat. pSim25-Ends construct was synthesized by ZAO “Eurogene” (Moscow).

DNA of simian adenovirus serotype 25 isolated from the virions was mixed with pSim25-Ends. Homologous recombination between pSim25-Ends and viral DNA resulted in plasmid pSim25-d1E1, which carries the genome of simian adenovirus serotype 25 with E1 site deleted.

Then from the constructed plasmid pSim25-d1E1 E3 site of adenovirus genome (3921 b.p. from the beginning of gene 12.5K to gene 14.7K) was deleted using conventional genetic engineering methods to increase the vector packing capacity. This resulted in plasmid construct pSim25-null encoding the full-length genome of simian adenovirus serotype 25 with deleted E1 an E3 sites of the genome. SEQ ID NO:6 was used as a maternal sequence of simian adenovirus serotype 25.

In addition, the authors developed several designs of the expression cassette:

expression cassette SEQ ID NO:4 comprises CMV promoter, gene encoding S protein of SARS-CoV-2 and polyadenylation signal;

expression cassette SEQ ID NO:2 comprises CAG promoter, gene encoding S protein of SARS-CoV-2 and polyadenylation signal;

expression cassette SEQ ID NO:3 comprises EF1 promoter, gene encoding S protein of SARS-CoV-2 and polyadenylation signal.

On the basis of plasmid construct pSim25-Ends the constructs pArms-Sim25-CMV-S-CoV2, pArms-Sim25-CAG-S-CoV2, pArms-Sim25-EF1-S-CoV2, containing expression cassettes SEQ ID NO:4, SEQ ID NO:2 or SEQ ID NO:3, respectively, and also bearing homology arms of the genome of simian adenovirus serotype 25 were obtained using the genetic engineering methods. Then the constructs pArms-Sim25-CMV-S-CoV2, pArms-Sim25-CAG-S-CoV2, pArms-Sim25-EF1-S-CoV2 were linearized at the unique hydrolysis site between the homology arms, each plasmid was mixed with recombinant vector pSim25-null. Homologous recombination resulted in recombinant plasmid vectors pSim25-CMV-S-CoV2, pSim25-CAG-S-CoV2, pSim25-EF1-S-CoV2, containing full-length genome of simian adenovirus serotype 25 with E1 and E3 sites deleted, and expression cassette SEQ ID NO:4, SEQ ID NO:2 or SEQ ID NO:3, respectively.

At Stage 3 the plasmids pSim25-CMV-S-CoV2, pSim25-CAG-S-CoV2, pSim25-EF1-S-CoV2 were hydrolyzed with specific restriction endonuclease to remove the vector part. The obtained DNA products were used for transfection of the cell culture HEK293. Resultant material was used for accumulation of recombinant adenoviruses in preparative amount.

The work resulted in obtaining the human adenoviruses serotype 25, containng the gene encoding S protein of SARS-CoV-2: simAd25-CMV-S-CoV2 (containing expression cassette SEQ ID NO:4), simAd25-CAG-S-CoV2 (containing expression cassette SEQ ID NO:2), simAd25-EF1-S-CoV2 (containing expression cassette SEQ ID NO:3).

Thus, the expression vector was obtained, containing the genome of recombinant strain of simian adenovirus serotype 25, with E1 and E3 sites deleted from the genome, and with integrated expression cassette selected from SEQ ID NO:4, SEQ ID NO:2, SEQ ID NO:3.

### EXAMPLE 4

Obtaining of immunobiological agent in the form of expression vector based on the genome of recombinant strain of simian adenovirus serotype 25, in which E 1 and E3 sites are deleted from the genome and with integrated expression cassette selected from SEQ ID NO:1, SEQ ID NO:2, SEQ ID NO:3.

At this stage of the work the expression vectors obtained in Example 3 were purified using anion exchange and exclusion chromatography. Resultant suspension contained adenovirus particles in the buffer for liquid form of the agent HJIH in the buffer for lyophilized form of the agent.

Thus, the following immunobiological agents based on genome of recombinant strain of simian adenovirus serotype 25, with E1 and E3 sites deleted from the genome were obtained:

1. Immunobiological agent based on genome of recombinant strain of simian adenovirus serotype 25, with E1 and E3 sites deleted from the genome, with expression cassette containing CMV promoter, the gene encoding S protein of SARS-CoV-2 and polyadenylation signal, SEQ ID NO:1 (simAd25-CMV-S-CoV2) in the buffer for liquid form of the agent.

2. Immunobiological agent based on genome of recombinant strain of simian adenovirus serotype 25, with E1 and E3 sites deleted from the genome, with expression cassette containing CMV promoter, the gene encoding S protein of SARS-CoV-2 and polyadenylation signal, SEQ ID NO:1 (simAd25-CMV-S-CoV2) in the buffer for lyophilized form of the agent.

3. Immunobiological agent based on genome of recombinant strain of simian adenovirus serotype 25, with E1 and E3 sites deleted from the genome, with expression cassette containing CAG promoter, the gene encoding S protein of SARS-CoV-2 and polyadenylation signal, SEQ ID NO:2 (simAd25-CAG-S-CoV2) in the buffer for liquid form of the agent.

4. Immunobiological agent based on genome of recombinant strain of simian adenovirus serotype 25, with E1 and E3 sites deleted from the genome, with expression cassette containing CAG promoter, the gene encoding S protein of SARS-CoV-2 and polyadenylation signal, SEQ ID NO:2 (simAd25-CAG-S-CoV2) in the buffer for lyophilized form of the agent.

5. Immunobiological agent based on genome of recombinant strain of simian adenovirus serotype 25, with E1 and E3 sites deleted from the genome, with expression cassette containing EF1 promoter, the gene encoding S protein of SARS-CoV-2 and polyadenylation signal, SEQ ID NO:3 (simAd25-EF1-S-CoV2) in the buffer for liquid form of the agent.

6. Immunobiological agent based on genome of recombinant strain of simian adenovirus serotype 25, with E1 and E3 sites deleted from the genome, with expression cassette containing EF1 promoter, the gene encoding S protein of SARS-CoV-2 and polyadenylation signal, SEQ ID NO:3 (simAd25-EF1-S-CoV2) in the buffer for lyophilized form of the agent.

Each of provided immunobiological agents is a component 2 in variant 1 and a component 1 in variant 3 of the developed agent.

### EXAMPLE 5

Obtaining of expression vector containing the genome of recombinant strain of human adenovirus serotype 5.

At Stage 1 the design of plasmid construct pAd5-Ends carrying two sites homologous to the genome of human adenovirus serotype 5 (two homology arms) was developed. One homology arm is a beginning of human adenovirus serotype 5 (from the left inverted terminal repeat to E1 site) and the sequence including pIX protein of the viral genome. The second homology arm contains the nucleotide sequence after ORF3 of E4 site to the end of genome. pAd5-Ends construct was synthesized by ZAO “Eurogene” (Moscow).

DNA of human adenovirus serotype 5 isolated from the virions was mixed with pAd5-Ends construct. Homologous recombination between pAdS-Ends and viral DNA resulted in plasmid pAd5-d1E1, which carries the genome of human adenovirus serotype 5 with E1 site deleted.

Then E3 site of the adenovirus genome (about 2685 b.p. from the end of gene 12.5K to the beginning of U-exon sequence) was deleted from the constructed plasmid pAd5-d1E1 using conventional genetic engineering methods to increase the vector packing capacity. This resulted in recombinant plasmid vector pAd5-too-null based on genome of human adenovirus serotype 5 with E1 and E3 deleted from the genome. SEQ ID NO:7 was used as a maternal sequence of human adenovirus serotype 5.

In addition, the authors developed several designs of the expression cassette:

expression cassette SEQ ID NO:1 comprises CMV promoter, gene encoding S protein of SARS-CoV-2 and polyadenylation signal;

expression cassette SEQ ID NO:2 comprises CAG promoter, gene encoding S protein of SARS-CoV-2 and polyadenylation signal;

expression cassette SEQ ID NO:3 comprises EF1 promoter, gene encoding S protein of SARS-CoV-2 and polyadenylation signal.

Then on the basis of plasmid construct pAdS-Ends the constructs pArms-Ad5-CMV-S-CoV2, pArms-Ad5-CAG-S-CoV2, pArms-Ad5-EF1-S-CoV2 containing expression cassettes SEQ ID NO:1, SEQ ID NO:2 or SEQ ID NO:3, respectively, and also bearing homology arms of the genome of human adenovirus serotype 5 were obtained using the genetic engineering methods.

Then the constructs pArms-Ad5-CMV-S-CoV2, pArms-Ad5-CAG-S-CoV2, pArms-Ad5-EF1-S-CoV2 were linearized at the unique hydrolysis site between the homology arms, each plasmid was mixed with recombinant vector pAdS-too-null. Homologous recombination resulted in plasmids pAd5-too-CMV-S-CoV2, pAd5-too-GAC-S-CoV2, pAd5-too-EF1-S-CoV2, carrying the genome of recombinant strain of human adenovirus serotype 5 with E1 [symbol] E3 sites deleted from the genome and expression cassettes SEQ ID NO:1, SEQ ID NO:2 or SEQ ID NO: 3, respectively.

At Stage 4 the plasmids pAd5-too-CMV-S-CoV2, pAd5-too-GAC-S-CoV2, pAd5-too-EF1-S-CoV2 were hydrolyzed with specific restriction endonuclease to remove the vector part. The obtained DNA product were used for transfection of the cell culture HEK293. Resultant material was used for accumulation of recombinant adenoviruses in preparative amounts.

The work resulted in obtaining the human adenoviruses serotype 5, containing the gene encoding S protein of SARS-CoV-2: Ad5-CMV-S-CoV2 (containing expression cassette SEQ ID NO:1), Ad5-CAG-S-CoV2 (containing expression cassette SEQ ID NO:2), Ad5-EF1-S-CoV2 (containing expression cassette SEQ ID NO:3).

Thus, the expression vector was obtained, containing the genome of recombinant strain of human adenovirus serotype 5 with E1 and E3 sites deleted from the genome, with integrated expression cassette selected from SEQ ID NO:1, SEQ ID NO:2, SEQ ID NO:3.

### EXAMPLE 6

Obtaining of immunobiological agent in the form of expression vector based on the genome of recombinant strain of human adenovirus serotype 5 with E1 and E3 sites deleted from the genome, with integrated expression cassette selected from SEQ ID NO:1, SEQ ID NO:2, SEQ ID NO:3.

At this stage of the work the expression vectors obtained in Example 5 were purified using anion exchange and exclusion chromatography. Resultant suspension contained adenovirus particles in the buffer for liquid form of the agent or in the buffer for lyophilized form of the agent.

Thus, the following immunobiological agents based on genome of recombinant strain of human adenovirus serotype 5, with E1 and E3 sites deleted from the genome:

1. Immunobiological agent based on the genome of recombinant strain of human adenovirus serotype 5, with E1 and E3 sites deleted from the genome, with expression cassette containing CMV promoter, the gene encoding S protein of SARS-CoV-2 and polyadenylation signal, SEQ ID NO:1 (Ad5-CMV-S-CoV2) in the buffer for liquid form of the agent.

2. Immunobiological agent based on the genome of recombinant strain of human adenovirus serotype 5, with E1 and E3 sites deleted from the genome, with expression cassette containing CMV promoter, the gene encoding S protein of SARS-CoV-2 and polyadenylation signal, SEQ ID NO:1 (Ad5-CMV-S-CoV2) in the buffer for lyophilized form of the agent.

3. Immunobiological agent based on the genome of recombinant strain of human adenovirus serotype 5, with E1 and E3 sites deleted from the genome, with expression cassette containing CAG promoter, the gene encoding S protein of SARS-CoV-2 and polyadenylation signal, SEQ ID NO:2 (Ad5-CAG-S-CoV2) in the buffer for liquid form of the agent.

4. Immunobiological agent based on the genome of recombinant strain of human adenovirus serotype 5, with E1 and E3 sites deleted from the genome, with expression cassette containing CAG promoter, the gene encoding S protein of SARS-CoV-2 and polyadenylation signal, SEQ ID NO:2 (Ad5-CAG-S-CoV2) in the buffer for lyophilized form of the agent.

5. Immunobiological agent based on the genome of recombinant strain of human adenovirus serotype 5, with E1 and E3 sites deleted from the genome, with expression cassette containing EF1 promoter, the gene encoding S protein of SARS-CoV-2 and polyadenylation signal, SEQ ID NO:3 (Ad5-EF1-S-CoV2) in the buffer for liquid form of the agent.

6. Immunobiological agent based on the genome of recombinant strain of human adenovirus serotype 5, with E1 and E3 sites deleted from the genome, with expression cassette containing EF1 promoter, the gene encoding S protein of SARS-CoV-2 and polyadenylation signal, SEQ ID NO:3 (Ad5-EF1-S-CoV2) in the buffer for lyophilized form of the agent.

Each of provided immunobiological agents is a component 1 in variant 1 and in variant 2 of the developed agent.

Each of provided immunobiological agents is a component 2 in variant 1 and in variant 3 of the developed agent.

### EXAMPLE 7

Preparation of buffer solution.

The developed agent according to the claimed invention comprises two components placed in separate vials. Each component is an immunobiological agent based on recombinant adenovirus with expression cassette in buffer solution.

The authors of the invention elaborated the composition of buffer solution to ensure stability of recombinant adenovirus particles. Said solution includes:

1. Tris(hydroxymethyl)aminomethane (Tris), which is required for maintaining pH of the solution.

2. Sodium chloride, which is added to achieve appropriate ionic strength and osmolarity.

3. Saccharose, which is used as cryoprotector.

4. Magnesium chloride hexahydrate, which is required as the source of bivalent cation.

5. EDTA, which is used as inhibitor of free-radical oxidation.

6. Polysorbate-80, which is used as a surfactant.

7. Ethanol 95%, which is used as inhibitor of free-radical oxidation.

8. Water, which is used as a solvent.

The author of the invention developed 2 variants of buffer solution for liquid form of the agent and for lyophilized form of the agent.

Several variants of experimental groups were obtained for determining the concentration of the compounds in the composition of buffer solution for liquid form of the agent (table 1). One of the components of the agent was added to each of the prepared buffer solutions:

1. Immunobiological agent based on recombinant human adenovirus serotype 26 with expression cassette containing CMV promoter, the gene encoding S protein of SARS-CoV-2, and polyadenylation signal, 1*10¹¹ virus particles.

2. Immunobiological agent based on recombinant human adenovirus serotype 5 with expression cassette containing CMV promoter, the gene encoding S protein of SARS-CoV-2, and polyadenylation signal, 1*10¹¹ virus particles.

3. Immunobiological agent based on recombinant simian adenovirus serotype 25 with expression cassette containing CMV promoter, the gene encoding S protein of SARS-CoV-2, and polyadenylation signal, 1*10¹¹ virus particles.

Thus, the stability of each of adenovirus serotypes in the agent composition was tested. Prepared pharmaceutical products were stored at the temperature −18° C. and −70 ° C. for 3 months, followed by thawing, and the change in recombinant adenovirus titer was assessed.

TABLE 1
Composition of experimental buffer solutions for liquid form of the agent.

| | Composition of buffer solution | | | | | | | |
|---|---|---|---|---|---|---|---|---|
| | | | | Magnesium | | | | |
| | | Sodium | | chloride | | | | |
| Group | Tris | chloride | Saccharose | hexahydrate | EDTA | Polysorbate- | Ethanol | |
| No | (mg) | (mg) | (mg) | (mg) | (mg) | 80 (mg) | 95% (mg) | Water |
| 1 | 0.968 | 2.19 | 25 | 0.102 | 0.019 | 0.25 | 0.0025 | up to 0.5 mL |
| 2 | 1.815 | 2.19 | 25 | 0.102 | 0.019 | 0.25 | 0.0025 | up to 0.5 mL |
| 3 | 1.21 | 1.752 | 25 | 0.102 | 0.019 | 0.25 | 0.0025 | up to 0.5 mL |
| 4 | 1.21 | 3.285 | 25 | 0.102 | 0.019 | 0.25 | 0.0025 | up to 0.5 mL |
| 5 | 1.21 | 2.19 | 20 | 0.102 | 0.019 | 0.25 | 0.0025 | up to 0.5 mL |
| 6 | 1.21 | 2.19 | 37.5 | 0.102 | 0.019 | 0.25 | 0.0025 | up to 0.5 mL |
| 7 | 1.21 | 2.19 | 25 | 0.0816 | 0.019 | 0.25 | 0.0025 | up to 0.5 mL |
| 8 | 1.21 | 2.19 | 25 | 0.153 | 0.019 | 0.25 | 0.0025 | up to 0.5 mL |
| 9 | 1.21 | 2.19 | 25 | 0.102 | 0.0152 | 0.25 | 0.0025 | up to 0.5 mL |
| 10 | 1.21 | 2.19 | 25 | 0.102 | 0.0285 | 0.25 | 0.0025 | up to 0.5 mL |
| 11 | 1.21 | 2.19 | 25 | 0.102 | 0.019 | 0.2 | 0.0025 | up to 0.5 mL |
| 12 | 1.21 | 2.19 | 25 | 0.102 | 0.019 | 0.375 | 0.0025 | up to 0.5 mL |
| 13 | 1.21 | 2.19 | 25 | 0.102 | 0.019 | 0.25 | 0.002 | up to 0.5 mL |
| 14 | 1.21 | 2.19 | 25 | 0.102 | 0.019 | 0.25 | 0.00375 | up to 0.5 mL |
| 15 | 1.21 | 2.19 | 25 | 0.102 | 0.019 | 0.25 | 0.0025 | up to 0.5 mL |

The results of the experiment showed that the titer of recombinant adenoviruses did not change after the storage in the buffer for liquid form of the agent at the temperature −18° C. and −70° C. for 3 months.

Therefore, the developed buffer solution for liquid form of the agent provides stability of all components of the developed agent in the following ranges of active ingredients (% by weight):

Tris: from 0.1831% by weight to 0.3432% by weight;

Sodium chloride: from 0.3313% by weight to 0.6212% by weight;

Saccharose: from 3,7821% by weight to 7,0915% by weight;

Magnesium chloride hexahydrate: from 0.0154% by weight to 0.0289% by weight;

EDTA: from 0.0029% by weight to 0.0054% by weight;

Polysorbate-80: from 0.0378% by weight to 0.0709% by weight;

Ethanol 95%: from 0.0004% by weight to 0.0007% by weight;

Solvent: balance.

Several variants of experimental groups were obtained for determining the concentration of the compounds in the composition of buffer solution for lyophilized form of the agent (table 2). One of the components of the agent was added to each of the prepared buffer solutions:

1. Immunobiological agent based on recombinant human adenovirus serotype 26 with expression cassette containing CMV promoter, the gene encoding S protein of SARS-CoV-2, and polyadenylation signal, 1*10¹¹ virus particles.

2. Immunobiological agent based on recombinant human adenovirus serotype 5 with expression cassette containing CMV promoter, the gene encoding S protein of SARS-CoV-2, and polyadenylation signal, 1*10¹¹ virus particles.

3. Immunobiological agent based on recombinant simian adenovirus serotype 25 with expression cassette containing CMV promoter, the gene encoding S protein of SARS-CoV-2 and polyadenylation signal, 1*10¹¹virus particles.

Thus, the stability of each of adenovirus serotypes in the agent composition was tested. Prepared pharmaceutical products were stored at the temperature +2 and +8° C. ° C. for 3 months, followed by thawing, and the change in recombinant adenovirus titer was assessed.

TABLE 2
Composition of experimental buffer solutions.

| | Composition of buffer solution | | | | | | |
|---|---|---|---|---|---|---|---|
| | | Sodium | | | | | |
| Group | Tris | chloride | Saccharose | Magnesium chloride | EDTA | Polysorbate- | |
| No | (mg) | (mg) | (mg) | hexahydrate (mg) | (mg) | 80 (mg) | Water |
| 1 | 0.1936 | 1.403 | 73.5 | 0.0204 | 0.0038 | 0.05 | up to 1 mL |
| 2 | 0.363 | 1.403 | 73.5 | 0.0204 | 0.0038 | 0.05 | up to 1 mL |
| 3 | 0.242 | 1.1224 | 73.5 | 0.0204 | 0.0038 | 0.05 | up to 1 mL |
| 4 | 0.242 | 2.1045 | 73.5 | 0.0204 | 0.0038 | 0.05 | up to 1 mL |
| 5 | 0.242 | 1.403 | 58.8 | 0.0204 | 0.0038 | 0.05 | up to 1 mL |
| 6 | 0.242 | 1.403 | 110.25 | 0.0204 | 0.0038 | 0.05 | up to 1 mL |
| 7 | 0.242 | 1.403 | 73.5 | 0.01632 | 0.0038 | 0.05 | up to 1 mL |
| 8 | 0.242 | 1.403 | 73.5 | 0.0306 | 0.0038 | 0.05 | up to 1 mL |
| 9 | 0.242 | 1.403 | 73.5 | 0.0204 | 0.00304 | 0.05 | up to 1 mL |
| 10 | 0.242 | 1.403 | 73.5 | 0.0204 | 0.0057 | 0.05 | up to 1 mL |
| 11 | 0.242 | 1.403 | 73.5 | 0.0204 | 0.0038 | 0.04 | up to 1 mL |
| 12 | 0.242 | 1.403 | 73.5 | 0.0204 | 0.0038 | 0.075 | up to 1 mL |
| 13 | 0.242 | 1.403 | 73.5 | 0.0204 | 0.0038 | 0.05 | up to 1 mL |

The results of the experiment showed that the titer of recombinant adenoviruses did not change after the storage in the buffer for lyophilized form of the agent at the temperature +2° C. [symbol] +8° C. for 3 months.

Therefore, the developed buffer solution for lyophilized form of the agent provides stability of all components of the developed agent in the following ranges of active ingredients (% by weight):

Tris: from 0.0180% by weight to 0.0338% by weight;

Sodium chloride: from 0.1044% by weight to 0.1957% by weight;

Saccharose: from 5,4688% by weight to 10.2539% by weight;

Magnesium chloride hexahydrate: from 0.0015% by weight to 0.0028% by weight;

EDTA: from 0.0003% by weight to 0.0005% by weight;

Polysorbate-80: from 0.0037% by weight to 0.0070% by weight;

Solvent: balance.

### EXAMPLE 8

The study of the developed agent immunogenicity through assessment of cell-mediated immune response against the antigen of SARS-CoV-2 virus in the blood of volunteers at different time intervals after vaccination.

The clinical studies of the develop agent, variant 1, included investigation of the intensity cell-mediated immunity.

40 volunteers participating in the study were immunized with:

1) Liquid form of the developed agent, variant 1: component 1 followed by component 2 within 21 days (component 1: Ad26-CMV-S-CoV2, component 2: Ad5-CMV-S-CoV2), at the dose of 1×10¹¹ virus particles (20 volunteers).

2) Lyophilized form of the developed agent, variant 1: component 1 followed by component 2 within 21 days (component 1: Ad26-CMV-S-CoV2, component 2: Ad5-CMV-S-CoV2), at the dose of 1×10¹¹ virus particles (20 volunteers).

On Day 0 (before the agent administration), Day 14 and Day 28 the blood samples were collected from the volunteers and centrifugated in the ficcol density gradient in order to isolate mononuclear cells. The isolated cells were stained with CFSE fluorescent dye (Invivogen, CIIIA) and added into the plate wells. Then the lymphocytes were restimulated in vitro by addition of coronavirus S protein into the culture medium (up to final protein concentration 1 μg/mL). Intact cells without antigen addition served as a negative control. 72 hours after antigen addition the percentage of proliferating cells was measured, and the culture medium was collected for measuring the interferon gamma level.

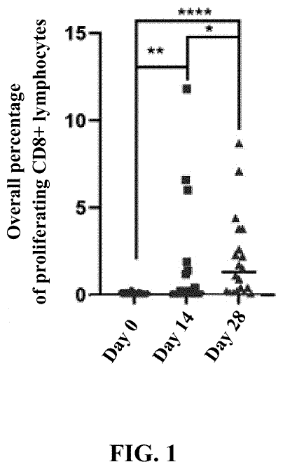

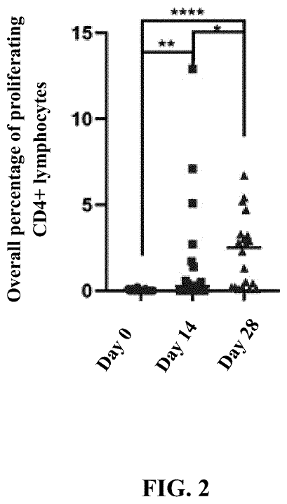

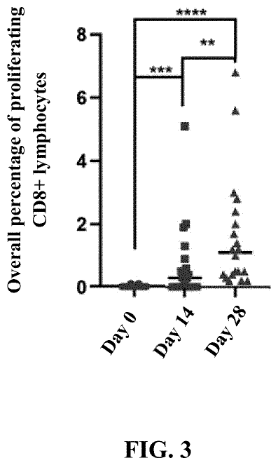

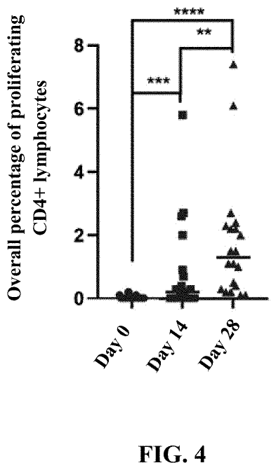

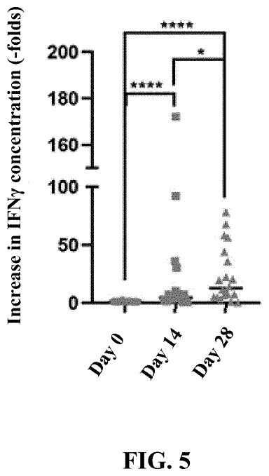

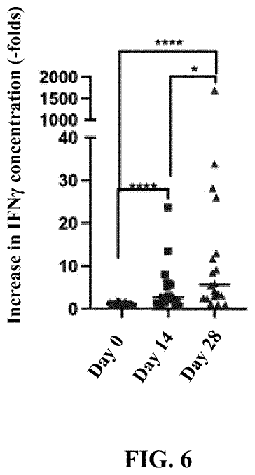

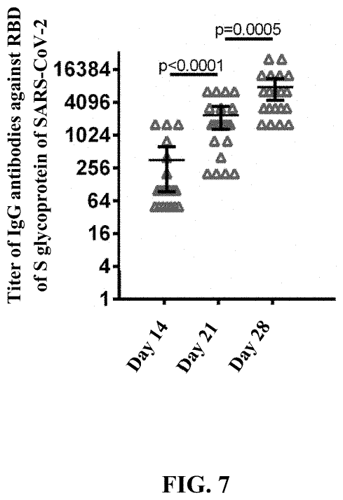

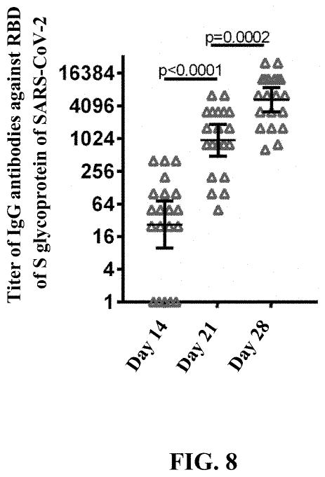

The cells were stained with antibodies against T cell marker molecules CD3, CD4, CD8 (anti-CD3 Pe-Cy7 (BD Biosciences, clone SK7), anti-CD4 APC (BD Biosciences, clone SK3), anti-CD8 PerCP-Cy5.5 (BD Biosciences, clone SK1)) to assess the percentage of proliferating cells. Flow cytofluorimeter BD FACS AriaIII (BD Biosciences, USA) was used to identify proliferating (carrying lesser amount of CFSE dye) CD4+ [symbol] CD8+ T cells in the cell mixture. The result obtained from analysis of the intact cells was subtracted from the result obtained from analysis of the cells restimulated with coronavirus antigen S in order to determine the resultant percentage of proliferating cells in each sample. Final results are shown in FIG. 1,2 (for liquid form of the vaccine) and FIG. 3,4 (for lyophilized form of the vaccine).

The concentration of interferon gamma (IFNγ) in the culture medium of human blood mononuclear cells was measured within 72 hours after restimulation with coronavirus S protein with the use of Interferon gamma EIA-BEST kit (VECTOR BEST, Russia) in accordance with instruction of manufacturer. The results are shown in FIG. 5 (for liquid form of the vaccine), and in FIG. 6 (for lyophilized form of the vaccine).

The study results showed that the intensity of cell-mediated immunity induced by consecutive immunization of volunteers with both forms of the agent, variant 1, grew with time elapsed from immunization, as evidenced by median percentage of proliferating CD4+ [symbol] CD8+ T cells. In both groups maximum values of proliferating CD4+ and CD8+ T cells were observed on Day 28 after immunization. Maximum statistically significant difference in percentages of proliferating CD4+ [symbol] CD8+ T cells (p<0.001) was observed between Day 0 and Day 28.

One can conclude from the results shown in FIG. 5,6 that based on the median increment of IFNγ concentration, the growth of intensity of cell-mediated immunity caused by consecutive immunization of volunteers with both forms of the agent, variant 1, was more pronounced with greater number of days after immunization. Statistically significant difference in IFNγ concentration increment between the pre-immunization level (Day 0) and Day 14 after vaccination was p<0.001. Maximum increment of IFNγ concentration is observed on Day 28 after immunization. Maximum statistically significant difference in IFNγ concentration increment (p<0.001) was observed between Day 0 and Day 28 of the study.

Thus, based on the above data one can conclude that immunization with the developed agent induces strong antigen-specific cell-mediated component of antiinfective immunity, which is supported by high statistical significance of the measured parameters before and after immunization.

### EXAMPLE 9

The study of the developed agent immunogenicity through assessing the titer of antibodies against SARS-CoV-2 antigen in the blood of volunteers at different time intervals after vaccination.

40 volunteers participating in the study were immunized with:

1) Liquid form of the developed agent, variant 1: component 1 followed by component 2 within 21 days (component 1: Ad26-CMV-S-CoV2, component 2: Ad5-CMV-S-CoV2), at the dose of 1×10¹¹ virus particles (20 volunteers).

2) Lyophilized form of the developed agent, variant 1: component 1 followed by component 2 within 21 days (component 1: Ad26-CMV-S-CoV2, component 2: Ad5-CMV-S-CoV2), at the dose of 1×10¹¹ virus particles (20 volunteers).

On Day 14, Day 21 and Day 28 the blood samples were collected, followed by serum separation.

The titer of antibodies against RBD of SARS-CoV-2 S protein was measured with the use of kit SARS-CoV-2-RBD-E1A-Gamaleya in accordance with instruction of manufacturer.

The results of assay of antibody titer against SARS-CoV-2 antigen in serum of volunteers after administration of liquid form of the agent are shown in FIG. 7.

The results of assay of antibody titer against SARS-CoV-2 antigen in serum of volunteers after administration of lyophilized form of the agent are shown in FIG. 8.

As is evident from the presented data, immunization of volunteers with the developed agent both in liquids and lyophilized form enables to generate strong (statistically significantly differing from the values obtained in non-immunized control group of animals) antibody-mediated immunity, which is characterized by increase in antibody level against S protein of SARS-CoV-2. The growth of intensity of antibody-mediated immune response with increasing time after immunization is observed.

### EXAMPLE 10

The use of the developed agent for prolongation of postvaccinal immunity against SARS-CoV-2 after immunization with the model subunit vaccine.

The objective of this study was to assess potential use of the developed agent for revaccination of the animals, which were immunized with the model subunit vaccine.

In this experiment female mice Balb/c with the body weight 18 g were used. At Stage 1 the animals were immunized with the model vaccine containing S protein of SARS-CoV-2 (10 μg/mouse) in phosphate-buffered saline with aluminium hydroxide (100 μg/mouse). Two doses of the vaccine were administered at 21 day interval between the doses. On Day 180 the animals were re-immunized with various variants of the developed agent. In the event of two-component agent: the first component (10¹⁰ virus particles/mouse) was administered on Day 180 of the experiment, and the second component (10¹⁰ v.p./mouse) was administered on Day 201. In the event of monocomponent agent immunization was carried out on Day 201 of the experiment. Thus, the following experimental and control group of animals were studied:

1) Model vaccine/Ad26-CMV-S-CoV2/Ad5-CMV-S-CoV2

2) Model vaccine/Ad26-CAG-S-CoV2/Ad5-CAG-S-CoV2

3) Model vaccine/Ad26-EF1-S-CoV2/Ad5-EF1-S-CoV2

4) Model vaccine/Ad26-CMV-S-CoV2/simAd25-CMV-S-CoV2

5) Model vaccine/Ad26-CAG-S-CoV2/simAd25-CAG-S-CoV2

6) Model vaccine/Ad26-EF1-S-CoV2/simAd25-EF1-S-CoV2

7) Model vaccine/simAd25-CMV-S-CoV2/Ad5-CMV-S-CoV2

8) Model vaccine/simAd25-CAG-S-CoV2/Ad5-CAG-S-CoV2

9) Model vaccine/simAd25-EF1-S-CoV2/Ad5-EF1-S-CoV2

10) Model vaccine/Ad26-CMV-S-CoV2

11) Model vaccine/Ad26-CAG-S-CoV2

12) Model vaccine/Ad26-EF1-S-CoV2

13) Model vaccine/Ad5-CMV-S-CoV2

14) Model vaccine/Ad5-CAG-S-CoV2

15) Model vaccine/Ad5-EF1-S-CoV2

16) Model vaccine/simAd25-CMV-S-CoV2

17) Model vaccine/simAd25-CAG-S-CoV2

18) Model vaccine/simAd25-EF1-S-CoV2

On Day 21, Day 180, and Day 222 of the experiment the blood from the tail vein was collected followed by serum separation. The titer of anti-SARS-CoV-2 antibodies was determined by enzyme immunoassay (EIA) according to the following protocol:

1) Antigen was adsorbed on the wells of 96-well microtitration plate at temperature +4° C. for 16 hours.

2) In order to preclude non-specific binding, the plate was “locked” with blocking buffer, which was added in each well in amount 100 μL/well. The plate was incubated on shaker at +37° C. for 1 hour.

3) The sera of immunized mice were diluted 100-fold and then a series of 2-fold dilutions was prepared.

4) 50 μL of each diluted serum sample was added into the plate wells.

5) Then the plate was incubated at +37° C. for 1 hour.

6) After the end of incubation the wells were washed with three portions of the phosphate buffer.

7) Then horseradish peroxidase-conjugated secondary antimouse-IgG antibodies were added.

8) Then the plate was incubated at +37° C. for 1 hour.

9) After the end of incubation the wells were washed with three portions of the phosphate buffer.

10) Then tetramethylbenzidine (TMB) solution was added, which is a horseradish substrate and turns into colored compound in the course of reaction. Within 15 minutes sulfuric acid was added to stop the reaction. Then optical density (OD) of solution was measured at wave length 450 nm in each well using spectrophotometer.

The antibody titer was determined as the highest dilution showing the solution optical density significantly greater than that in the negative control group. The results (geometrical means) are shown in table 3.

TABLE 3
Titer of anti-S protein antibodies in the murine serum
(geometrical mean antibody titer).
Table 3

| | | Day of Experiment | | |
|---|---|---|---|---|
| | | Day 21 | Day 180 | Day 222 |
| 1 | Model vaccine/Ad26- | 1393 | 606 | 117627 |
| | CMV-S-CoV2/Ad5-CMV- | | | |
| | S-CoV2 | | | |
| 2 | Model vaccine/Ad26- | 1600 | 528 | 89144 |
| | CAG-S-CoV2/Ad5-CAG - | | | |
| | S-CoV2 | | | |
| 3 | Model vaccine/Ad26- EF1- | 1213 | 696 | 102400 |
| | S-CoV2/Ad5- EF1-S-CoV2 | | | |
| 4 | Model vaccine/Ad26- | 1600 | 606 | 58813 |
| | CMV-S-CoV2/simAd25- | | | |
| | CMV-S-CoV2 | | | |
| 5 | Model vaccine/Ad26- | 1393 | 528 | 67559 |
| | CAG-S-CoV2/simAd25- | | | |
| | CAG-S-CoV2 | | | |
| 6 | Model vaccine/Ad26-EF1- | 1393 | 528 | 58813 |
| | S-CoV2/simAd25-EF1-S- | | | |
| | CoV2 | | | |
| 7 | Model vaccine/simAd25- | 1600 | 606 | 89144 |
| | CMV-S-CoV2/Ad5-CMV- | | | |
| | S-CoV2 | | | |
| 8 | Model vaccine/simAd25- | 1213 | 606 | 89144 |
| | CAG-S-CoV2/Ad5-CAG - | | | |
| | S-CoV2 | | | |
| 9 | Model vaccine/simAd25- | 1393 | 696 | 102400 |
| | EF1-S-CoV2/Ad5-EF1-S- | | | |
| | CoV2 | | | |
| 10 | Model vaccine/Ad26- | 1600 | 528 | 16890 |
| | CMV-S-CoV2 | | | |
| 11 | Model vaccine/Ad26- | 1600 | 528 | 19401 |
| | CAG-S-CoV2 | | | |
| 12 | Model vaccine/Ad26-EF1- | 1600 | 696 | 19401 |
| | S-CoV2 | | | |
| 13 | Model vaccine/Ad5-CMV- | 1600 | 606 | 51200 |
| | S-CoV2 | | | |
| 14 | Model vaccine/Ad5-CAG- | 1600 | 606 | 44572 |
| | S-CoV2 | | | |
| 15 | Model vaccine/Ad5-EF1- | 1600 | 528 | 51200 |
| | S-CoV2 | | | |
| 16 | Model vaccine/simAd25- | 1600 | 606 | 33779 |
| | CMV-S-CoV2 | | | |
| 17 | Model vaccine/simAd25- | 1393 | 528 | 38802 |
| | CAG-S-CoV2 | | | |
| 18 | Model vaccine/simAd25- | 1600 | 528 | 33779 |
| | EF1-S-CoV2 | | | |

The presented data demonstrate the development of antibodies in all animals after immunization with model inactivated vaccine; by Day 180 after immunization the antibody titers decrease, however revaccination of the animals with various variants of the developed agent results in manifold increase in blood antibody titer. Thus, the experimental data support the use of the developed agent for prolongation of postvaccinal immunity against SARS-CoV-2.

### EXAMPLE 11

The use of the developed agent for prolongation of postvaccinal immunity against SARS-CoV-2 after immunization with model inactivated vaccine.

The objective of this study was to assess potential use of the developed agent for revaccination of the animals, which were immunized with the model inactivated vaccine.

In this experiment female mice Balb/c with the body weight 18 g were used. At Stage 1 the animals were immunized with the model vaccine containing formalin-inactivated SARS-CoV-2 virus. Two doses of the vaccine were administered at 21 day interval between the doses. On Day 180 the animals were re-immunized with various variants of the developed agent. In the event of two-component agent: the first component (10¹⁰ v.p./mouse) was administered on Day 180 of the experiment, and the second component (10¹⁰ v.p./mouse) was administered on Day 201. In the event of monocomponent agent immunization was carried out on Day 201 of the experiment. Thus, the following experimental and control group of animals were studied:
1. Model vaccine/Ad26-CMV-S-CoV2/Ad5-CMV-S-CoV2
2. Model vaccine/Ad26-CAG-S-CoV2/Ad5-CAG-S-CoV2
3. Model vaccine/Ad26-EF1-S-CoV2/Ad5-EF1-S-CoV2
4. Model vaccine/Ad26-CMV-S-CoV2/simAd25-CMV-S-CoV2
5. Model vaccine/Ad26-CAG-S-CoV2/simAd25-CAG-S-CoV2
6. Model vaccine/Ad26-EF1-S-CoV2/simAd25-EF1-S-CoV2
7. Model vaccine/simAd25-CMV-S-CoV2/Ad5-CMV-S-CoV2
8. Model vaccine/simAd25-CAG-S-CoV2/Ad5-CAG-S-CoV2
9. Model vaccine/simAd25-EF1-S-CoV2/Ad5-EF1-S-CoV2
10. Model vaccine/Ad26-CMV-S-CoV2
11. Model vaccine/Ad26-CAG-S-CoV2
12. Model vaccine/Ad26-EF1-S-CoV2
13. Model vaccine/Ad5-CMV-S-CoV2
14. Model vaccine/Ad5-CAG-S-CoV2
15. Model vaccine/Ad5-EF1-S-CoV2
16. Model vaccine/simAd25-CMV-S-CoV2
17. Model vaccine/simAd25-CAG-S-CoV2
18. Model vaccine/simAd25-EF1-S-CoV2

On Day 21, Day 180, and Day 222 of the experiment the blood from the tail vein was collected followed by serum separation. The titer of anti-SARS-CoV-2 antibodies was determined by enzyme immunoassay (E1A) according to the following protocol:
1) Antigen was adsorbed on the wells of 96-well microtitration plate at temperature +4° C. for 16 hours.
2) In order to preclude non-specific binding, the plate was “locked” with blocking buffer, which was added in each well in amount 100 μL/well. The plate was incubated on shaker at +37° C. for 1 hour.
3) The sera of immunized mice were diluted 100-fold and then a series of 2-fold dilutions was prepared.
4) 50 μL of each diluted serum sample was added into the plate wells.
5) Then the plate was incubated at +37° C. for 1 hour.
6) After the end of incubation the wells were washed with three portions of phosphate buffer.
7) Then horseradish peroxidase-conjugated secondary antimouse-IgG antibodies were added.
8) Then the plate was incubated at +37° C. for 1 hour.
9) After the end of incubation the wells were washed with three portions of the phosphate buffer.
10) Then tetramethylbenzidine (TMB) solution was added, which is a horseradish substrate and turns into colored compound in the course of reaction. Within 15 minutes sulfuric acid was added to stop the reaction. Then optical density (OD) of solution was measured at wave length 450 nm in each well using spectrophotometer.

The antibody titer was determined as the highest dilution showing the solution optical density significantly greater than that in the negative control group. The results (geometrical means) are shown in table 4.

TABLE 4
Titer of anti-S protein antibodies in the murine serum
(geometrical mean antibody titer).
Table 4

| | | Day of Experiment | | |
|---|---|---|---|---|
| | | Day 21 | Day 180 | Day 222 |
| 1 | Model vaccine/Ad26-CMV-S-CoV2/ | 303 | 200 | 135118 |
| | Ad5-CMV-S-CoV2 | | | |
| 2 | Model vaccine/Ad26-CAG-S-CoV2/ | 348 | 152 | 117627 |
| | Ad5-CAG-S-CoV2 | | | |
| 3 | Model vaccine/Ad26-EF1-S-CoV2/ | 264 | 174 | 155209 |
| | Ad5-EF1-S-CoV2 | | | |
| 4 | Model vaccine/Ad26-CMV-S-CoV2/ | 303 | 115 | 58813 |
| | simAd25-CMV-S-CoV2 | | | |
| 5 | Model vaccine/Ad26-CAG-S-CoV2/ | 264 | 174 | 77605 |
| | simAd25-CAG-S-CoV2 | | | |
| 6 | Model vaccine/Ad26-EF1-S-CoV2/ | 303 | 174 | 67559 |
| | simAd25-EF1-S-CoV2 | | | |
| 7 | Model vaccine/simAd25-CMV-S- | 264 | 115 | 135118 |
| | CoV2/Ad5-CMV-S-CoV2 | | | |
| 8 | Model vaccine/simAd25-CAG-S- | 264 | 174 | 117627 |
| | CoV2/Ad5-CAG-S-CoV2 | | | |
| 9 | Model vaccine/simAd25-EF1-S-CoV2/ | 303 | 115 | 117627 |
| | Ad5-EF1-S-CoV2 | | | |
| 10 | Model vaccine/Ad26-CMV-S-CoV2 | 264 | 174 | 19401 |
| 11 | Model vaccine/Ad26-CAG-S-CoV2 | 348 | 132 | 19401 |
| 12 | Model vaccine/Ad26-EF1-S-CoV2 | 303 | 174 | 16890 |
| 13 | Model vaccine/Ad5-CMV-S-CoV2 | 303 | 132 | 58813 |
| 14 | Model vaccine/Ad5-CAG-S-CoV2 | 264 | 152 | 58813 |
| 15 | Model vaccine/Ad5-EF1-S-CoV2 | 348 | 174 | 51200 |
| 16 | Model vaccine/simAd25-CMV-S- | 348 | 152 | 38802 |
| | CoV2 | | | |
| 17 | Model vaccine/simAd25-CAG-S- | 264 | 152 | 44572 |
| | CoV2 | | | |
| 18 | Model vaccine/simAd25-EF1-S-CoV2 | 303 | 132 | 38802 |

The presented data demonstrate the development of antibodies in all animals after immunization with model inactivated vaccine; by Day 180 after immunization the antibody titers decrease, however revaccination of the animals with various variants of the developed agent results in manifold increase in blood antibody titer. Thus, the experimental data support the use of the developed agent for prolongation of postvaccinal immunity against SARS-CoV-2.

### EXAMPLE 12

The use of the developed agent for prolongation of postvaccinal immunity against SARS-CoV-2 after immunization with various variants of the developed agent.

The objective of this study was to assess potential use of the developed agent for revaccination of the animals, which were immunized with various variants of the developed agent.

In this experiment female mice Balb/c with the body weight 18 g were used. At Stage 1 the animals were immunized with various monocomponent variants of the developed agent (10¹⁰ v.p./mouse). On Day 180 the animals were re-immunized with various two-component variants of the developed agent. (10¹⁰ v.p./mouse). The first component (10¹⁰ v.p./mouse) was administered on Day 180 of the experiment, and the second component (10¹⁰ v.p./mouse) was administered on Day 201. In the event of monocomponent agent immunization was carried out on Day 201 of the experiment. Thus, the following experimental and control group of animals were studied:
1) Ad26-CMV-S-CoV2/Ad26-CMV-S-CoV2, Ad5-CMV-S-CoV2
2) Ad26-CMV-S-CoV2/Ad26-CMV-S-CoV2, simAd25-CMV-S-CoV2
3) Ad26-CMV-S-CoV2/simAd25-CMV-S-CoV2, Ad5-CMV-S-CoV2
4) Ad5-CMV-S-CoV2/Ad26-CMV-S-CoV2, Ad5-CMV-S-CoV2
5) Ad5-CMV-S-CoV2/Ad26-CMV-S-CoV2, simAd25-CMV-S-CoV2
6) Ad5-CMV-S-CoV2/simAd25-CMV-S-CoV2, Ad5-CMV-S-CoV2
7) simAd25-CMV-S-CoV2/Ad26-CMV-S-CoV2, Ad5-CMV-S-CoV2
8) simAd25-CMV-S-CoV2/Ad26-CMV-S-CoV2, simAd25-CMV-S-CoV2
9) simAd25-CMV-S-CoV2/simAd25-CMV-S-CoV2, Ad5-CMV-S-CoV2
10) Ad26-CMV-S-CoV2/Ad26-CMV-S-CoV2
11) Ad26-CMV-S-CoV2/Ad5-CMV-S-CoV2
12) Ad26-CMV-S-CoV2/simAd25-CMV-S-CoV2
13) Ad5-CMV-S-CoV2/Ad26-CMV-S-CoV2
14) Ad5-CMV-S-CoV2/Ad5 -CMV-S-CoV2
15) Ad5-CMV-S-CoV2/simAd25-CMV-S-CoV2
16) simAd25-CMV-S-CoV2/Ad26-CMV-S-CoV2
17) simAd25-CMV-S-CoV2/Ad5-CMV-S-CoV2
18) simAd25-CMV-S-CoV2/simAd25-CMV-S-CoV2

On Day 21, Day 180, and Day 222 of the experiment the blood from the tail vein was collected followed by serum separation. The titer of anti-SARS-CoV-2 antibodies was determined by enzyme immunoassay (EIA) according to the following protocol:
1) Antigen was adsorbed on the wells of 96-well microtitration plate at temperature +4° C. for 16 hours.
2) In order to preclude non-specific binding, the plate was “locked” with blocking buffer, which was added in each well in amount 100 μL/well. The plate was incubated on shaker at +37° C. for 1 hour.
3) The sera of immunized mice were diluted 100-fold and then a series of 2-fold dilutions was prepared.
4) 50 μL of each diluted serum sample was added into the plate wells.
5) Then the plate was incubated at +37° C. for 1 hour.
6) After the end of incubation the wells were washed with three portions of the phosphate buffer.
7) Then horseradish peroxidase-conjugated secondary antimouse-IgG antibodies were added.
8) Then the plate was incubated at +37° C. for 1 hour.
9) After the end of incubation the wells were washed with three portions of phosphate buffer.
10) Then tetramethylbenzidine (TMB) solution was added, which is a horseradish substrate and turns into colored compound in the course of reaction. Within 15 minutes sulfuric acid was added to stop the reaction. Then optical density (OD) of solution was measured at wave length 450 nm in each well using spectrophotometer.

The antibody titer was determined as the highest dilution showing the solution optical density significantly greater than that in the negative control group. The results (geometrical means) are shown in table 5.

TABLE 5
Titer of anti-S protein antibodies in the murine serum
(geometrical mean antibody titer).
Table 5

| | | Day of Experiment | | |
|---|---|---|---|---|
| | | Day 21 | Day 180 | Day 222 |
| 1 | Ad26-CMV-S-CoV2/Ad26-CMV-S- | 2786 | 2111 | 204800 |
| | CoV2, Ad5-CMV-S-CoV2 | | | |
| 2 | Ad26-CMV-S-CoV2/Ad26-CMV-S- | 2425 | 2425 | 155209 |
| | CoV2, simAd25-CMV-S-CoV2 | | | |
| 3 | Ad26-CMV-S-CoV2/simAd25-CMV- | 2111 | 1838 | 178289 |
| | S-CoV2, Ad5-CMV-S-CoV2 | | | |
| 4 | Ad5-CMV-S-CoV2/Ad26-CMV-S- | 29407 | 25600 | 155209 |
| | CoV2, Ad5-CMV-S-CoV2 | | | |
| 5 | Ad5-CMV-S-CoV2/Ad26-CMV-S- | 33779 | 29407 | 178289 |
| | CoV2, simAd25-CMV-S-CoV2 | | | |
| 6 | Ad5-CMV-S-CoV2/simAd25-CMV- | 38802 | 33779 | 155209 |
| | S-CoV2, Ad5-CMV-S-CoV2 | | | |
| 7 | simAd25-CMV-S-CoV2/Ad26-CMV- | 12800 | 9701 | 155209 |
| | S-CoV2, Ad5-CMV-S-CoV2 | | | |
| 8 | simAd25-CMV-S-CoV2/Ad26-CMV- | 14703 | 11143 | 178289 |
| | S-CoV2, simAd25-CMV-S-CoV2 | | | |
| 9 | simAd25-CMV-S-CoV2/simAd25- | 16890 | 11143 | 155209 |
| | CMV-S-CoV2, Ad5-CMV-S-CoV2 | | | |
| 10 | Ad26-CMV-S-CoV2/Ad26-CMV-S- | 3200 | 2111 | 117627 |
| | CoV2 | | | |
| 11 | Ad26-CMV-S-CoV2/Ad5-CMV-S- | 2425 | 2111 | 102400 |
| | CoV2 | | | |
| 12 | Ad26-CMV-S-CoV2/simAd25-CMV- | 2425 | 2111 | 117627 |
| | S-CoV2 | | | |
| 13 | Ad5-CMV-S-CoV2/Ad26-CMV-S- | 25600 | 22286 | 102400 |
| | CoV2 | | | |
| 14 | Ad5-CMV-S-CoV2/Ad5-CMV-S- | 29407 | 25600 | 117627 |
| | CoV2 | | | |
| 15 | Ad5-CMV-S-CoV2/simAd25-CMV- | 38802 | 33779 | 102400 |
| | S-CoV2 | | | |
| 16 | simAd25-CMV-S-CoV2/Ad26-CMV- | 16890 | 12800 | 117627 |
| | S-CoV2 | | | |
| 17 | simAd25-CMV-S-CoV2/Ad5-CMV- | 16890 | 14703 | 117627 |
| | S-CoV2 | | | |
| 18 | simAd25-CMV-S-CoV2/simAd25- | 19401 | 16890 | 102400 |
| | CMV-S-CoV2 | | | |

The presented data demonstrate the development of antibodies in all animals after immunization of mice with monocomponent variants of the developed agent; by Day 180 after immunization the antibody titers decrease, however revaccination of the animals with various variants of the developed agent results in manifold increase in blood antibody titer. Thus, the experimental data support the use of the developed agent for prolongation of postvaccinal immunity against SARS-CoV-2.

### EXAMPLE 13.

The use of the developed agent for prolongation of postvaccinal immunity against SARS-CoV-2 after immunization with various variants of the developed agent.

The objective of this study was to assess potential use of the developed agent for revaccination of the animals, which were immunized with various variants of the developed agent.

In this experiment female mice Balb/c with the body weight 18 g were used. At Stage 1 the animals were immunized with variants of the developed two-component agent (10¹⁰ v.p./mouse) at 21 day interval. On Day 180 the animals were re-immunized with various monocomponent variants of the developed agent. Thus, the following experimental and control group of animals were studied:
1) Ad26-CMV-S-CoV2, Ad5-CMV-S-CoV2/Ad26-CMV-S-CoV2
2) Ad26-CMV-S-CoV2, Ad5-CMV-S-CoV2/Ad5-CMV-S-CoV2
3) Ad26-CMV-S-CoV2, Ad5-CMV-S-CoV2/simAd25-CMV-S-CoV2
4) Ad26-CMV-S-CoV2, simAd25-CMV-S-CoV2/Ad26-CMV-S-CoV2
5) Ad26-CMV-S-CoV2, simAd25-CMV-S-CoV2/Ad5-CMV-S-CoV2
6) Ad26-CMV-S-CoV2, simAd25-CMV-S-CoV2/simAd25-CMV-S-CoV2
7) simAd25-CMV-S-CoV2, Ad5-CMV-S-CoV2/Ad26-CMV-S-CoV2
8) simAd25-CMV-S-CoV2, Ad5-CMV-S-CoV2/Ad5-CMV-S-CoV2
9) simAd25-CMV-S-CoV2, Ad5-CMV-S-CoV2/simAd25-CMV-S-CoV2
10) Ad26-CMV-S-CoV2, Ad5-CMV-S-CoV2/Ad26-CMV-S-CoV2, Ad5-CMV-S-CoV2
11) Ad26-CMV-S-CoV2, simAd25-CMV-S-CoV2/Ad26-CMV-S-CoV2, simAd25-CMV-S-CoV2
12) simAd25-CMV-S-CoV2, Ad5-CMV-S-CoV2/simAd25-CMV-S-CoV2, Ad5-CMV-S-CoV2
13) Ad26-CMV-S-CoV2, Ad5-CMV-S-CoV2/Ad26-CMV-S-CoV2, simAd25-CMV-S-CoV2

On Day 42, Day 180, and Day 222 of the experiment the blood from the tail vein was collected followed by serum separation. The titer of anti-SARS-CoV-2 antibodies was determined by enzyme immunoassay (EIA) according to the following protocol:
1) Antigen was adsorbed on the wells of 96-well microtitration plate at temperature +4° C. for 16 hours.
2) In order to preclude non-specific binding, the plate was “locked” with blocking buffer, which was added in each well in amount 100 μL/well. The plate was incubated on shaker at +37° C. for 1 hour.
3) The sera of immunized mice were diluted 100-fold and then a series of 2-fold dilutions was prepared.
4) 50 μL of each diluted serum sample was added into the plate wells.
5) Then the plate was incubated at +37° C. for 1 hour.
6) After the end of incubation the wells were washed with three portions of the phosphate buffer.
7) Then horseradish peroxidase-conjugated secondary antimouse-IgG antibodies were added.
8) Then the plate was incubated at +37° C. for 1 hour.
9) After the end of incubation the wells were washed with three portions of phosphate buffer.
10) Then tetramethylbenzidine (TMB) solution was added, which is a horseradish substrate and turns into colored compound in the course of reaction. Within 15 minutes sulfuric acid was added to stop the reaction. Then optical density (OD) of solution was measured at wave length 450 nm in each well using spectrophotometer.

The antibody titer was determined as the highest dilution showing the solution optical density significantly greater than that in the negative control group. The results (geometrical means) are shown in table 6.

TABLE 6
Titer of anti-S protein antibodies in the murine serum
(geometrical mean antibody titer).
Table 6

| | | Day of experiment | | |
|---|---|---|---|---|
| | | Day 21 | Day 180 | Day 222 |
| 1 | Ad26-CMV-S-CoV2/Ad26-CMV-S- | 44572 | 38802 | 204800 |
| | CoV2, Ad5-CMV-S-CoV2 | | | |
| 2 | Ad26-CMV-S-CoV2/Ad26-CMV-S- | 38802 | 33779 | 178289 |
| | CoV2, simAd25-CMV-S-CoV2 | | | |
| 3 | Ad26-CMV-S-CoV2/simAd25- | 51200 | 38802 | 204800 |
| | CMV-S-CoV2, Ad5-CMV-S-CoV2 | | | |
| 4 | Ad5-CMV-S-CoV2/Ad26-CMV-S- | 33779 | 29407 | 178289 |
| | CoV2, Ad5-CMV-S-CoV2 | | | |
| 5 | Ad5-CMV-S-CoV2/Ad26-CMV-S- | 29407 | 25600 | 178289 |
| | CoV2, simAd25-CMV-S-CoV2 | | | |
| 6 | Ad5-CMV-S-CoV2/simAd25-CMV- | 29407 | 25600 | 178289 |
| | S-CoV2, Ad5-CMV-S-CoV2 | | | |
| 7 | simAd25-CMV-S-CoV2/Ad26- | 38802 | 33779 | 178289 |
| | CMV-S-CoV2, Ad5-CMV-S-CoV2 | | | |
| 8 | simAd25-CMV-S-CoV2/Ad26- | 38802 | 33779 | 204800 |
| | CMV-S-CoV2, simAd25-CMV-S- | | | |
| | CoV2 | | | |
| 9 | simAd25-CMV-S-CoV2/simAd25- | 33779 | 33779 | 155209 |
| | CMV-S-CoV2, Ad5-CMV-S-CoV2 | | | |
| 10 | Ad26-CMV-S-CoV2, Ad5-CMV-S- | 44572 | 38802 | 204800 |
| | CoV2/Ad26-CMV-S-CoV2 | | | |
| 11 | Ad26-CMV-S-CoV2, Ad5-CMV-S- | 33779 | 29407 | 204800 |
| | CoV2/Ad5-CMV-S-CoV2 | | | |
| 12 | Ad26-CMV-S-CoV2, Ad5-CMV-S- | 38802 | 33779 | 204800 |
| | CoV2/simAd25-CMV-S-CoV2 | | | |
| 13 | Ad26-CMV-S-CoV2, simAd25- | 38802 | 33779 | 178289 |
| | CMV-S-CoV2/Ad26-CMV-S-CoV2 | | | |
| 14 | Ad26-CMV-S-CoV2, simAd25- | 44572 | 38802 | 204800 |
| | CMV-S-CoV2/Ad5-CMV-S-CoV2 | | | |
| 15 | Ad26-CMV-S-CoV2, simAd25- | 38802 | 33779 | 178289 |
| | CMV-S-CoV2/simAd25-CMV-S- | | | |
| | CoV2 | | | |
| 16 | simAd25-CMV-S-CoV2, Ad5-CMV- | 51200 | 38802 | 204800 |
| | S-CoV2/Ad26-CMV-S-CoV2 | | | |
| 17 | simAd25-CMV-S-CoV2, Ad5-CMV- | 33779 | 29407 | 178289 |
| | S-CoV2/simAd25-CMV-S-CoV2 | | | |
| 18 | simAd25-CMV-S-CoV2, Ad5-CMV- | 29407 | 25600 | 178289 |
| | S-CoV2/simAd25-CMV-S-CoV2 | | | |

The presented data demonstrate the development of antibodies in all animals after immunization of mice with two-component variants of the developed agent; by Day 180 after immunization the antibody titers decrease, however revaccination of the animals with various variants of the developed agent results in manifold increase in blood antibody titer. Thus, the experimental data support the use of the developed agent for prolongation of postvaccinal immunity against SARS-CoV-2.

Thus, the assigned technical problem, specifically, creation of the agents providing prolongation of postvaccinal immunity against SARS-CoV-2 virus, is solved as supported by presented examples.

### Industrial Use

All presented examples support the efficacy of the agents, which provide efficacious induction of immune response and also prolongation of postvaccinal immunity against SARS-CoV-2 virus and industrial use.

1. A method for revaccination against a disease caused by severe acute respiratory syndrome virus (SARS-CoV-2), comprising:
administering an agent to a subject, the agent comprising in the form of a first component comprising an expression vector based on a genome of recombinant strain of human adenovirus serotype 26, wherein the E1 and E3 sites are deleted from the genome and the site ORF6-Ad26 is substituted for ORF6-Ad5 with an integrated expression cassette selected from SEQ ID NO:1, SEQ ID NO:2, or SEQ ID NO:3; and/or
a second component comprising an expression vector based on a genome of recombinant strain of human adenovirus serotype 5 with E1 and E3 sites deleted from the genome with uan integrated expression cassette selected from SEQ ID NO:1, SEQ ID NO:2, or SEQ ID NO:3.

2. A method for revaccination against a disease caused by severe acute respiratory syndrome virus (SARS-CoV-2), comprising:
administering an agent to a subject, the agent comprising;
a first component comprising an expression vector based on a genome of recombinant strain of human adenovirus serotype 26 wherein the E1 and E3 sites are deleted from the genome, and the site ORF6-Ad26 is substituted for ORF6-Ad5 with an integrated expression cassette selected from SEQ ID NO:1, SEQ ID NO:2, or SEQ ID NO:3; and/or
a second component comprising an expression vector based on a genome of recombinant strain of simian adenovirus serotype 25 wherein the E1 and E3 sites are deleted from the genome with integrated expression cassette selected from SEQ ID NO:4, SEQ ID NO:2, or SEQ ID NO:3.

3. A method for revaccination against a disease caused by severe acute respiratory syndrome virus (SARS-CoV-2), comprising:
administering an agent to a subject, the agent comprising:
a first component expression vector based on a genome of recombinant strain of simian adenovirus serotype 25 wherein the E1 and E3 sites are deleted from the genome with integrated expression cassette selected from SEQ ID NO:4, SEQ ID NO:2, or SEQ ID NO:3; and/or
a second component comprising an expression vector based on a genome of recombinant strain of human adenovirus serotype 5 wherein the E1 and E3 sites are deleted from the genome with the integrated expression cassette selected from SEQ ID NO:1, SEQ ID NO:2, or SEQ ID NO:3.

4. The method of claim 1, wherein the agent is in a liquid or lyophilized form.

5. The method of claim 4, wherein a buffer for the liquid form comprises, by weight percent(%):

| | |
|---|---|
| tris | 0.1831 to 0.3432 |
| sodium chloride | 0.3313 to 0.6212 |
| saccharose | 3.7821 to 7.0915 |
| magnesium chloride hexahydrate | 0.0154 to 0.0289 |
| EDTA | 0.0029 to 0.0054 |
| polysorbate 80 | 0.0378 to 0.0709 |
| ethanol 95% | 0.0004 to 0.0007 |
| water | balance. |

6. The method of claim 4, wherein the lyophilized form is reconstituted and comprises a buffer composed of, by weight percent (%):

| | |
|---|---|
| tris | 0.0180 to 0.0338 |
| sodium chloride | 0.1044 to 0.1957 |
| saccharose | 5.4688 to 10.2539 |
| magnesium chloride hexahydrate | 0.0015 to 0.0028 |
| EDTA | 0.0003 to 0.0005 |
| polysorbate 80 | 0.0037 to 0.0070 |
| water | balance. |

7. The method of claim 1, wherein the first component and the second component are in separate containers.

8. The method of claim 2, wherein the agent is in a liquid or lyophilized form.

9. The method of claim 8, wherein a buffer for the liquid form comprises, by weight percent(%):

| | |
|---|---|
| tris | 0.1831 to 0.3432 |
| sodium chloride | 0.3313 to 0.6212 |
| saccharose | 3.7821 to 7.0915 |
| magnesium chloride hexahydrate | 0.0154 to 0.0289 |
| EDTA | 0.0029 to 0.0054 |
| polysorbate 80 | 0.0378 to 0.0709 |
| ethanol 95% | 0.0004 to 0.0007 |
| water | balance. |

10. The method of claim 8, wherein the lyophilized form is reconstituted and comprises a buffer composed of, by weight percent (%):

| | |
|---|---|
| tris | 0.0180 to 0.0338 |
| sodium chloride | 0.1044 to 0.1957 |
| saccharose | 5.4688 to 10.2539 |
| magnesium chloride hexahydrate | 0.0015 to 0.0028 |
| EDTA | 0.0003 to 0.0005 |
| polysorbate 80 | 0.0037 to 0.0070 |
| water | balance. |

11. The method of claim 2, wherein the first component and the second component are in separate containers.

12. The method of claim 3, wherein the agent is in a liquid or lyophilized form.

13. The method of claim 12, wherein a buffer for the liquid form comprises, by weight percent(%):

| | |
|---|---|
| tris | 0.1831 to 0.3432 |
| sodium chloride | 0.3313 to 0.6212 |
| saccharose | 3.7821 to 7.0915 |
| magnesium chloride hexahydrate | 0.0154 to 0.0289 |
| EDTA | 0.0029 to 0.0054 |
| polysorbate 80 | 0.0378 to 0.0709 |
| ethanol 95% | 0.0004 to 0.0007 |
| water | balance. |

14. The method of claim 12, wherein the lyophilized form is reconstituted and comprises a buffer composed of, by weight percent (%):

| | |
|---|---|
| tris | 0.0180 to 0.0338 |
| sodium chloride | 0.1044 to 0.1957 |
| saccharose | 5.4688 to 10.2539 |
| magnesium chloride hexahydrate | 0.0015 to 0.0028 |
| EDTA | 0.0003 to 0.0005 |
| polysorbate 80 | 0.0037 to 0.0070 |
| water | balance |

15. The method of claim 3, wherein the first component and the second component are in separate containers.
